SEQUENCE LISTING (50 sequences)

SEQ ID NO: 1 - protein, 54 aa, Homo sapiens
MLQMAGQCSQNEYFDSLLHACIPCQLRCSSNTPPLTCQRYCNASVTNSVKGTNA

SEQ ID NO: 2 - protein, 123 aa, synthetic construct
MLQMAGQCSQNEYFDSLLHACIPCQLRCSSNTPPLTCQRYCNASVTNSVKGTNAGGGSGGGSPRGSGGGSMLQMAGQCSQNEYFDSLLHACIPCQLRCSSNTPPLTCQRYCNASVTNSVKGTN

SEQ ID NO: 3 - protein, 272 aa, synthetic construct
PEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWK

SEQ ID NO: 4 - protein, 272 aa, synthetic construct
PEEPLVVKVEEGDEAWLPCLKGTSDGPTQQLTWSRESPLKPFLKVSFGVPGLGVHVRPNAVSLVISQVSQQMGGFYLCQPGPPSEKAWQPGWTVNVEGSGELFRWQVSDLGGLGCGLKQRSSEGPSSPSGKLMSPKLYVWAKDRPEIWEGEPPCLPPRDSLQQSLSRDMTVAPGSTLWLSCGVPPDSVSRGPLSWTHVHPKGPKSLLSLELKDDRPARDMWVTGTRLFLPRATAQDAGKYYCHRGQLTMSFHLEVKARPVSAHTKLRTGGWK

SEQ ID NO: 5 - protein, 272 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWK

SEQ ID NO: 6 - protein, 47 aa, Homo sapiens
KISHFLKMESLNFIRAHTPYINIYNCEPANPSEKNSPSTQYCYSIQS

SEQ ID NO: 7 - protein, 668 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 8 - protein, 204 aa, Homo sapiens
SGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSM

SEQ ID NO: 9 - protein, 147 aa, Homo sapiens
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 10 - protein, 499 aa, Homo sapiens
QPSVSPGEPSPPSIHPGKSDLIVRVGDEIRLLCTDPGFVKWTFEILDETNENKQNEWITEKAEATNTGKYTCTNKHGLSNSIYVFVRDPAKLFLVDRSLYGKEDNDTLVRCPLTDPEVTNYSLKGCQGKPLPKDLRFIPDPKAGIMIKSVKRAYHRLCLHCSVDQEGKSVLSEKFILKVRPAFKAVPVVSVSKASYLLREGEEFTVTCTIKDVSSSVYSTWKRENSQTKLQEKYNSWHHGDFNYERQATLTISSARVNDSGVFMCYANNTFGSANVTTTLEVVDKGFINIFPMINTTVFVNDGENVDLIVEYEAFPKPEHQQWIYMNRTFTDKWEDYPKSENESNIRYVSELHLTRLKGTEGGTYTFLVSNSDVNAAIAFNVYVNTKPEILTYDRLVNGMLQCVAAGFPEPTIDWYFCPGTEQRCSASVLPVDVQTLNSSGPPFGKLVVQSSIDSSAFKHNGTVECKAYNDVGKTSAYFNFAFKGNNKEQIHPHTLFTP

SEQ ID NO: 11 - protein, 258 aa, Homo sapiens
VPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 12 - protein, 53 aa, Homo sapiens
DQWSTQDLYNNPVTAVFNYQGLWRSCVRESSGFTECRGYFTLLGLPAMLQAVR

SEQ ID NO: 13 - protein, 31 aa, Homo sapiens
VTNFWMSTANMYTGMGGMVQTVQTRYTFGAA

SEQ ID NO: 14 - protein, 248 aa, Homo sapiens
HTAAQDNCTCPTNKMTVCSPDGPGGRCQCRALGSGMAVDCSTLTSKCLLLKARMSAPKNARTLVRPSEHALVDNDGLYDPDCDPEGRFKARQCNQTSVCWCVNSVGVRRTDKGDLSLRCDELVRTHHILIDLRHRPTAGAFNHSDLDAELRRLFRERYRLHPKFVAAVHYEQPTIQIELRQNTSQKAAGDVDIGDAAYYFERDIKGESLFQGRGGLDLRVRGEPLQVERTLIYYLDEIPPKFSMKRLT

SEQ ID NO: 15 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 16 - protein, 17 aa, Homo sapiens
IPNGYLVLDLSMQEALS

SEQ ID NO: 17 - protein, 11 aa, Homo sapiens
YXVXDLSMQEL

SEQ ID NO: 18 - protein, 5 aa, synthetic construct
LPXTG

SEQ ID NO: 19 - protein, 121 aa, Homo sapiens
EPKSPKSADKTHTAPQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 113 aa, Homo sapiens
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 21 - protein, 5 aa, synthetic construct
GGGGX

SEQ ID NO: 22 - protein, 6 aa, synthetic construct
GSSGSX

SEQ ID NO: 23 - protein, 5 aa, synthetic construct
GGGGS

SEQ ID NO: 24 - protein, 5 aa, synthetic construct
GSPRG

SEQ ID NO: 25 - protein, 10 aa, synthetic construct
GGGGSGGGGS

SEQ ID NO: 26 - protein, 15 aa, synthetic construct
GGGGSGGGGSGGGGS

SEQ ID NO: 27 - protein, 20 aa, synthetic construct
GGGGSGGGGSGGGGSGGGGS

SEQ ID NO: 28 - protein, 19 aa, synthetic construct
GSPRGGGGSGGGGSGGGGS

SEQ ID NO: 29 - protein, 18 aa, synthetic construct
GSTSGSGKPGSGEGSTKG

SEQ ID NO: 30 - protein, 18 aa, synthetic construct
KESGSVSSEQLAQFRSLD

SEQ ID NO: 31 - protein, 14 aa, synthetic construct
EGKSSGSGSESKST

SEQ ID NO: 32 - protein, 12 aa, synthetic construct
GSAGSAAGSGEF

SEQ ID NO: 33 - protein, 9 aa, Homo sapiens
QSFGLLDPK

SEQ ID NO: 34 - protein, 23 aa, Homo sapiens
LSEGDKVKMDSRIQVLSRGVNQT

SEQ ID NO: 35 - protein, 49 aa, Homo sapiens
KPTTTPAPRPPTPAPTIASQPLSKRPEACRPAAGGAVHTRGLDFACDIY

SEQ ID NO: 36 - protein, 39 aa, Homo sapiens
IEVMYPPPYLDNERSNGTIIHVKGKHLCPSPLFPGPSKP

SEQ ID NO: 37 - protein, 22 aa, Homo sapiens
EPKSCDKTHTCPPCPAPELLGG

SEQ ID NO: 38 - protein, 18 aa, Homo sapiens
ERKCCVECPPCPAPPAAA

SEQ ID NO: 39 - protein, 19 aa, Homo sapiens
ESKYGPPCPPCPAPEFLGG

SEQ ID NO: 40 - protein, 6 aa, synthetic construct
DTYRYI

SEQ ID NO: 41 - protein, 6 aa, synthetic construct
TDFYLK

SEQ ID NO: 42 - protein, 6 aa, synthetic construct
HHHHHH

SEQ ID NO: 43 - protein, 8 aa, synthetic construct
DYKDDDDK

SEQ ID NO: 44 - protein, 24 aa, synthetic construct
EFDYKDDDDKGSLPETGGHHHHHH

SEQ ID NO: 45 - protein, 19 aa, synthetic construct
KDHLIHNVHKEFHAHAHNK

SEQ ID NO: 46 - protein, 8 aa, synthetic construct
QPELAPED

SEQ ID NO: 47 - protein, 5 aa, synthetic construct
LPETG

SEQ ID NO: 48 - protein, 295 aa, synthetic construct
GSPEEPLVVKVEEGDEAWLPCLKGTSDGPTQQLTWSRESPLKPFLKVSFGVPGLGVHVRPNAVSLVISNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSRDMTVAPGSTLWLSCGVPPDSVSRGPLSWTHVHPKGPKSLLSLELKDDRPARDMWVTGTRLFLPRATAQDAGKYYCHRGNLTMSFHLEVKARPVSAHTKLRTGGWKGSDYKDDDDKLPETGHHHHHH

SEQ ID NO: 49 - DNA, 30 bp, synthetic construct
  source 1..30: mol_type = other DNA
ccaccacccagctttcttgtacaaagtggt

SEQ ID NO: 50 - DNA, 37 bp, synthetic construct
  source 1..37: mol_type = other DNA
tcaggcggccgcgaccagggctctttgaagatgaaga

(19) United States
(12) Patent Application Publication
(10) Pub. No.: US 2026/0306681 A1
(43) Pub. Date: Oct. 8, 2026

(54) DEVELOPMENT OF A PLATFORM TO NONINVASIVELY IMAGE CAR T CELLS

(71) Applicants: Ali Salehi FARID, Boston, MA (US); Jennifer ROWLEY, Boston, MA (US); Michael HEMANN, Cambridge, MA (US); Julia FRÖSE, Cambridge, MA (US); DANA-FARBER CANCER INSTITUTE, INC., Boston, MA (US); MASSACHUSETTS INSTITUTE OF TECHNOLOGY, Cambridge, MA (US)

(72) Inventors: Ali Salehi FARID, Boston, MA (US); Jennifer ROWLEY, Boston, MA (US); Michael HEMANN, Cambridge, MA (US); Julia FRÖSE, Cambridge, MA (US); Mohammad RASHIDIAN, Boston, MA (US); Taha RAKHSHANDEHROO, Boston, MA (US)

(73) Assignees: DANA-FARBER CANCER INSTITUTE, INC., Boston, MA (US); MASSACHUSETTS INSTITUTE OF TECHNOLOGY, Cambridge, MA (US)

(21) Appl. No.: 19/479,964

(22) PCT Filed: May 1, 2024
(86) PCT No.: PCT/US2024/027275

§ 371 Date: Oct. 29, 2025

Related U.S. Application Data
(60) Provisional application No. 63/463,405, filed on May 2, 2023.

(51) Int. Cl. A61K 51/08; A61K 51/06; A61K 51/10

(52) U.S. Cl. CPC A61K 51/088; A61K 51/065; A61K 51/10; A61K 2123/00

(57) ABSTRACT

Disclosed are radiolabeled tracers, diagnostic compositions, methods of imaging and diagnosing of CAR immune cell function in vivo, and methods of stimulating CAR immune cells and treating cancer.

### CROSS-REFERENCE TO RELATED APPLICATIONS

This application claims the benefit of priority under 35 U.S.C. § 119(e) to U.S. Provisional Application No. 63/463,405, filed May 2, 2023, which is incorporated herein by reference in its entirety.

### SEQUENCE LISTING

The instant application contains a Sequence Listing which has been submitted electronically in XML format and is hereby incorporated by reference in its entirety. Said XML copy, created on Apr. 24, 2024, is named 52095_777001WO_SL.xml and is 48 KB bytes in size.

### BACKGROUND OF THE DISCLOSURE

Chimeric Antigen Receptor (CAR) T cells have shown great promise in the treatment of a spectrum of blood-borne malignancies. A CAR combines a binding domain, often a single-chain variable fragment (scFv), that binds to a tumor-associated or tumor-specific antigen, and intracellular signaling motifs capable of T or NK cell activation (Feins et al., Am. J. Hematol. 94(S1):S3-S9 (2019)). In most cases, T cells expressing a CAR (CAR T cells) recognize structures present on a cancer cell's surface independent of a particular MHC molecule. MHC-independent antigen recognition enables CAR T cells to treat any patient whose tumor expresses the antigen. Many different antigens have been targeted by CAR T cells with some showing promise (Turtle et al., J. Clin. Invest. 126(6):2123-2138 (2016), Raje et al., N. Engl. J. Med. 380(18):1726-1737 (2019)), and several CAR T cell therapies targeting cluster of differentiation antigen (CD) 19 and B-cell maturation antigen (BCMA) are FDA-approved.

However, CAR T cell therapies often have heterogeneous responses where the CAR T cells do not persist in vivo, and the patients receiving the cells do not respond to the therapy. Techniques that monitor the dynamics and persistence of CAR T cells in vivo can be used to better understand and treat these heterogeneous responses (Hou et al., Dis. Markers 2019:3425291 (2019)). Current methods to assess CAR T cells in vivo include detection of cytokines (Hou et al., Dis. Markers 2019:3425291 (2019)), digital PCR analysis (Teachey et al., Cancer Discov. 6(6):664-679 (2016)), or flow cytometry on blood samples (Fehse et al., Mol. Ther. Methods Clin. Dev. 16:172-178 (2020)). However, these methods do not provide spatial information of infused CAR T cells, and the blood analyses may not accurately quantify the extent of CAR T cell tumor infiltration and expansion.

Imaging-based diagnoses may be able to detect CAR cells in vivo. Imaging-based techniques such as positron emission tomography (PET), single-photon emission computerized tomography (SPECT), and magnetic resonance imaging (MRI), may provide valuable data for predicting long-term outcomes, and associated toxicities such as cytopenias. However, common imaging techniques require either ex vivo radiolabeling of CAR cells before adoptive transfer (Zhou et al., J. Nucl. Med. 46(5):816-22 (2005), Weist et al., J. Nucl. Med. 59(10):1531-1537 (2018), Pittet et al., Proc. Natl. Acad. Sci. U.S.A 104(30):12457-61 (2007)), or genetic addition of a reporter gene into the CAR cells and concomitant use of an imaging radiolabeled tracer specific for the reporter gene (Gambhir et al., Neoplasia 2(1-2):118-38 (2000), Blasberg and Gelovani-Tjuvajev, J. Cell. Biochem. Suppl. 39:172-83 (2002), Yaghoubi and Gambhir, Nat. Protoc. 1(6):3069-75 (2006), Kang, et al., J. Nucl. Med. 49(Suppl 2):164S-79S (2008), Dobrenkov et al., J. Nucl. Med. 49(7):1162-70 (2008), Yaghoubi et al., Theranostics 2(4):374-91 (2012)). Ex vivo labeling requires rapid imaging (up to a few days post-injection) due to dilution and effluxion of the labeling agent, a major constraint to clinical applicability and in vivo detection of a reporter gene may result in immunogenicity to the reporter protein, background signal to physiologic expression in host tissues, promoter silencing, potential DNA modifications-induced mutations, and further design and operation complexities.

Therefore, these methods do not always correlate to a positive therapeutic response (Zheng et al., J. Transl. Med. 10:29 (2012)). Consequently, there is a critical need for methods that accurately correlate CAR T cell detection and the patient's response to the CAR-T therapy.

### SUMMARY OF THE DISCLOSURE

The radiolabeled tracers, diagnostic compositions, methods of detecting CAR immune cells, kits, and methods of treating cancer provided herein are expected to address the above needs. Significantly, they are applicable to FDA-approved CAR T cell therapies without requiring changes to established clinical protocols.

A first aspect of the present disclosure is directed to a radiolabeled tracer that contains an ectodomain of an antigen present on a cancer cell and a radioactive isotope detectable via positron emission tomography (PET) or single-photon emission computerized tomography (SPECT) connected to the ectodomain. The ectodomain of the radiolabeled tracer is designed to bind the extracellular domain of a CAR that targets the ectodomain of the antigen on a cancer cell.

Another aspect of the present disclosure is directed to a diagnostic composition that contains the radiolabelled tracer and a pharmaceutically acceptable carrier.

Another aspect of the present disclosure is directed to a method of detecting chimeric antigen receptor (CAR) immune cells in a subject. The method entails administering to the subject the diagnostic composition, wherein prior to the administering of the diagnostic composition, the subject has been administered an effective number of immune cells containing a CAR that contains an extracellular domain that binds the ectodomain of the radiolabeled tracer, a transmembrane domain, and an intracellular domain containing a stimulatory domain (CAR immune cells) and producing a first image of a body area of the subject by PET or SPECT imaging, wherein detecting the radioactive isotope in the body area is an indication of the CAR immune cells in the body area.

Yet another aspect of the present disclosure is directed to a kit containing (a) an effective amount of the radiolabeled tracer and (b) printed instructions for use in administering to a subject having received or receiving an administration of an effective number of CAR immune cells.

Yet another aspect of the present disclosure is directed to a method of treating cancer in a subject. The method entails determining the presence of CAR immune cells in a body area of a subject by administering to the subject the diagnostic composition, wherein prior to the administering of the diagnostic composition, the subject has been administered an effective number of immune cells containing a CAR that contains an extracellular domain that binds the ectodomain of the radiolabeled tracer, a transmembrane domain, and an intracellular domain containing a stimulatory domain (CAR immune cells), producing a first image of a body area of the subject by PET or SPECT imaging, wherein detecting the radioactive isotope in the body area is an indication of the CAR immune cells in the body area, and administering an appropriate therapy to the subject following a determination that the presence or number of CAR immune cells is suboptimal.

Working examples disclosed herein demonstrate that radiolabeled tracers that contain an ectodomain of a cancer antigen bind to CAR immune cells, and may be used by PET imaging to determine CAR immune cell localization in in vivo cancer models. The radiolabeled tracers disclosed herein may be used to guide improved treatments, allow optimization of the dose and timing of the CAR immune cell administration, and help avoid potential lethal systemic toxicity of the infused CAR immune cells.

### BRIEF DESCRIPTION OF THE DRAWINGS

FIG. 1A-FIG. 1J are a set of illustrations, photographs, and line graphs showing the strategy and results of imaging CAR T cells by radiolabeled tracers containing a cancer ectodomain and a radioactive isotope. FIG. 1A schematically illustrates a radiolabeled tracer bound to a CAR expressed on the surface of a CAR T cell. FIG. 1B is a set of line graphs that shows dose-dependent staining of CAR T cells containing CARs that bind CD19 or BCMA with Alexa Fluor 647 (AF647)-labeled tracers containing either the CD19 or BCMA cancer antigen ectodomain by flow cytometry. FIG. 1C is a set of line graphs that shows CAR T cell-mediated target cell killing with CAR T cells containing CARs that bind CD19 or BCMA with a range of doses of the tracers and target cells expressing either CD19 or BCMA. FIG. 1D is a set of line graphs that shows a lack of CAR T cell-mediated target cell killing with CAR T cells containing CARs that bind CD19 or BCMA with a range of doses of the tracers and target cells that do not express CD19 or BCMA. FIG. 1E and FIG. 1F are a set of photographs that show mice orthotopically implanted with human CD19⁺-PDAC-tumors and injected with CAR T cells. The mice in FIG. 1E received CAR T cells containing a CAR that binds CD19 and the mice in FIG. 1F received CAR T cells containing a CAR that binds EGFR (non-specific control). FIG. 1G schematically illustrates site-specific labeling of the human CD19 ectodomain with Alexa-647 fluorophore at the CD19 ectodomain C-terminus via a sortase reaction. FIG. 1H is a set of photographs that shows characterization of the CD19 tracer through SDS-PAGE analysis with Coomassie and Alexa647-fluorescence, right and left, respectively. Lanes include, #1: marker, #2: CD19, and #3: Alexa647-labeled CD19. FIG. 1I is a line graph that shows flow cytometric analysis histograms displaying specific staining of CD19 CAR T cells, but not control CAR T cells, with 10 nM Alexa647-labeled CD19 tracer. FIG. 1J is a line graph that shows dose-dependent percent CD19 tracer binding (left y-axis) and mean fluorescent intensity (MFI) (right y-axis) to CD19 CAR T cells (EC₅₀=0.074 nM) and control (EGFRvIII) CAR T cells.

FIG. 2A-FIG. 2D are a set of illustrations, flow cytometry, and bar graphs showing detection of CD19 CAR T cells by a CD19 tracer in vivo with high efficacy and specificity. FIG. 2A schematically illustrates the experimental design. FIG. 2B is a set of flow cytometry graphs that shows B-ALL cells and CAR T cells in blood, bone marrow, and spleen. CAR T cells stained with the tracer are shown in the boxes. Population numbers shown are in %. FIG. 2C is a bar graph that shows the efficiency of CD19 tracer binding to CD19 CAR T cells compared to control CAR T cells and other cells present in the analyzed organs. FIG. 2D is a bar graph that shows MFI of CD19 tracer binding to CD19 CAR T cells.

FIG. 3A-FIG. 3G are a set of line graphs showing the impact of the CD19 tracer on CAR T-cell activity. FIG. 3A-FIG. 3B are a set of line graphs that show cell counts and IFN-γ from CD19 CAR T cells or control CAR T cells incubation with hCD19⁺ B-ALL cells in the presence of CD19 tracer. FIG. 3C-FIG. 3D are a set of line graphs that show cell counts and IFN-γ from CD19 CAR T cells or control CAR T cells incubation with hCD19⁺ PDAC cells in the presence of CD19 tracer. FIG. 3E is a line graph that shows IFN-γ from CD19 CAR T cells or control CAR T cells incubation with hCD19⁻ B-ALL cells in the presence of CD19 tracer. FIG. 3F-FIG. 3G are a set of line graphs that shows cell counts and IFN-γ from CD19 CAR T cells or control CAR T cells incubation with hCD19⁻ PDAC cells in the presence of CD19 tracer.

FIG. 4A-FIG. 4D are a set of illustrations, photographs, bar, and line graphs showing detection of CD19 CAR T cells in vivo in a syngeneic mouse model of B-ALL. FIG. 4A schematically illustrates the experimental design. FIG. 4B is a set of PET-CT imaging photographs of mice 24 hours after the tracer injection. Photographs are, from left to right, whole mouse CT scan overlaid with PET signal, PET signal alone, and a transverse view of the mouse in CT scan only and PET/CT overlay at 24 hours post CD19 tracer injection. Arrows indicate the spleen in the PET images, and circles indicate the kidney (Kid.) and spleen (Spl.) in the transverse CT images. FIG. 4C is a bar graph that shows quantification of standardized uptake values (SUV) of the CD19 tracer in the spleen 24 hours after tracer injection. FIG. 4D is a line graph that shows survival analysis of mice categorized by CAR T cell treatment and spleen SUV values.

FIG. 5A-FIG. 5B are a set of flow cytometry and line graphs showing site-specific labeling and installation of a CD19 tracer and effects on specificity and binding efficacy. FIG. 5A is a set of flow cytometry graphs that shows CD19 tracer binding CAR T cells. FIG. 5B is a set of line graphs that shows dose-dependent tracer binding (left y-axis) and MFI (right y-axis) to CD19 CAR T cells and control (EGFRvIII) CAR T cells, using the DFO-PEG labeled CD19 tracer before and after Zirconium installation.

FIG. 6A-FIG. 6B are a set of flow cytometry graphs showing flow cytometric analysis of antigen expression on tumor cells co-cultured with CAR T cells in presence of CD19 tracer in vitro. FIG. 6A is a set of flow cytometric graphs that shows hCD19⁺ B-ALL cells incubated with CAR T cells (E:T ratio=10:1) and increasing concentrations of the CD19 tracer for 24 hours. FIG. 6B is a set of flow cytometric graphs that shows hCD19⁺ PDAC cells incubated with CAR T cells (E:T ratio=10:1) and increasing concentrations of the CD19 tracer for 24 hours.

FIG. 7A-FIG. 7D are a set of line graphs showing CD19 tracer binding CAR T cells in the presence of tumor cells during an in vitro killing assay. FIG. 7A-FIG. 7D are line plots that show dose-dependent analysis of hCD19⁻ A647 tracer binding (left y-axis) and MFI (right axis) to hCD19 CAR T cells and control (EGFRvIII) CAR T cells used during an in vitro killing assay with hCD19⁺ B-ALL cells (FIG. 7A), hCD19⁻ B-ALL cells (FIG. 7B), hCD19′ PDAC cells (FIG. 7C), and hCD19⁻ PDAC cells (FIG. 7D).

FIG. 8A-FIG. 8D are a set of illustrations, photographs, and line graphs showing no impact on CAR T-cell functionality in vivo from the CD19 tracer. FIG. 8A is a set of photographs that shows representative bioluminescence imaging in mice with luciferase-expressing B-ALL cells to daily monitor tumor burden during CAR T cell treatment. FIG. 8B is a line graph that shows quantitative analysis of bioluminescent imaging of B-ALL cells over time (n=5). FIG. 8C schematically illustrates the experimental design for B-ALL, CAR T cell, and tracer injections. FIG. 8D is a line graph that shows Survival monitoring of mice injected with B-ALL and CAR T cells alone or in combination with CAR T cells and the Zr-DFO-PEG-CD19 tracer until moribund state (n=4-5).

FIG. 9A-FIG. 9E are a set of illustrations, photographs, bar, and line graphs showing optimization and characterization of ⁸⁹Zr-CD19 PET tracer in vivo. FIG. 9A schematically illustrates the experimental design of B-ALL, CAR T-cell and tracer injections for PET imaging. FIG. 9B is a set of photographs that shows whole body PET/CT and PET images of CD19 CAR T-cells in B-ALL carrying mice using DFO-labeled CD19 tracer with PEG5, PEG10 and PEG20 moieties, 24 hours after tracer injection. All images have the same CT and PET settings for comparison. Spleen shown with blue arrow. FIG. 9C is a set of photographs that shows ex vivo PET images of spleens and bone marrow from FIG. 9B. FIG. 9D is a bar graph that shows biodistribution of the ⁸⁹Zr-DFO-PEG20-labeled CD19 tracer in all major organs in B-ALL carrying mice (n=4 to 8 for each group). FIG. 9E is a line graph that shows blood curve generated from serial sampling following ⁸⁹Zr-DFO-PEG20-labeled CD19 tail vein injection, measured with a gamma counter (n=4-5 for each group). The EC50 was calculated for tracer clearance from mice injected with control CAR T-cells (EC₅₀=103 min) or CD19 CAR T cells (EC₅₀=122 min); ID=Injected dose.

FIG. 10 is a set of photographs of PET-CT imaging showing detection of CD19 CAR T cells in vivo in a syngeneic mouse model of B-ALL 24 hours after the tracer injection, as schematically illustrated in FIG. 4A. Photographs are, from left to right, whole mouse CT scan overlaid with PET signal, PET signal alone, and a transverse view of the mouse in CT scan only and PET/CT overlay at 24 hours post CD19 tracer injection. Arrows indicate the spleen in the PET images, and circles indicate the kidney (Kid.) and spleen (Spl.) in the transverse CT images.

### DETAILED DESCRIPTION OF THE DISCLOSURE

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated to facilitate the understanding of the present disclosure.

As used in the description and the appended claims, the singular forms “a”, “an”, and “the” mean “one or more” and therefore include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The term “approximately” as used herein refers to a range of values that fall within 25%, 20%, 19%, 18% 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

The transitional term “comprising,” which is synonymous with “including,” “containing,” or “characterized by,” is inclusive or open-ended and does not exclude additional, unrecited elements or method steps. By contrast, the transitional phrase “consisting of” excludes any element or method step not specified in the claim (or the specific element or method step with which the phrase “consisting of” is associated). The transitional phrase “consisting essentially of” limits the scope of a claim to the specified elements and method or steps and “unrecited elements and method steps that do not materially affect the basic and novel characteristic(s)” of the claimed disclosure.

### Radiolabeled Tracer

In one aspect, the disclosure provides a radiolabeled tracer which contains an ectodomain of an antigen present on a cancer cell (also referred to herein as a cancer antigen) and a radioactive isotope detectable via positron emission tomography (PET) or single-photon emission computerized tomography (SPECT) connected to the ectodomain. The term “antigen” as used herein refers to a target protein expressed by a cancer (e.g., tumor) cell. The ectodomain of an antigen is at least a portion of the antigen that is exposed on the cancer cell surface. As known in the art, the term “cancer” as used herein refers to a disease characterized by uncontrolled cellular proliferation, reduced cellular apoptosis, and spread of abnormal cells that invade and destroy non-cancerous tissues.

The ectodomain of the radiolabeled tracer is designed to bind the extracellular domain of a chimeric antigen receptor (CAR) that targets the ectodomain of the cancer antigen. The CAR is presented on an immune cell, which is used with the radiolabeled tracer in the present methods.

In some embodiments, the radiolabeled tracer is formulated and administered as a monomeric protein. In other embodiments, the radiolabeled tracer is formulated and administered in the form of a homodimer.

### Ectodomains

The ectodomain of the radiolabeled tracer binds the extracellular domain of a CAR presented on an immune cell. As is known in the art, the ectodomain of a cancer antigen is the portion of the antigen on the surface of a cancer cell. CAR immune cells such as CAR T cells are designed to bind the ectodomains of cancer antigens. The ectodomain may be formed by contiguous or non-contiguous amino acid residues in the extracellular domain of a cancer antigen. In some embodiments, the radiolabeled tracer ectodomain includes the entire extracellular domain of a cancer antigen. In some embodiments, the radiolabeled tracer ectodomain includes a portion of an extracellular domain of a cancer antigen.

Ectodomains of cancer antigens are known in the art and/or may be readily identifiable in accordance with standard techniques. To confirm the desired activity, a putative ectodomain for use in the present radiolabeled tracer may be assayed for binding with (previously administered) CAR T cells that target the cancer antigen.

In some embodiments, the subject was previously administered CAR immune cells (e.g., CAR T-cells) that target B-cell maturation antigen (BCMA). CAR immune cells that target BCMA are known in the art. See, e.g., FDA-approved CAR-expressing immune cells ciltacabtagene autoleucel (Carvykti®), and idecabtagene vicleucel (Abecma®), U.S. Pat. Nos. 10,072,088, 10,683,369, 11,084,880, 10,174,095, and 11,186,647, and U.S. Patent Application Publication Nos. 2016/0131655, 2017/0226216, 2018/0133296, 2019/0151365, 2019/0359727, 2019/0381171, 2020/0339699, 2020/0360431, 2020/0055948, 2021/0128618, 2022/0033509, and 2022/0064316. Such CAR immune cells may include an extracellular domain obtainable from a commercially available anti-BCMA antibody, BCMA-binding fragment, or derivative thereof, e.g., belantamab (Blenrep®), linvoseltamab (REGN5458), pacanalotamab (AMG 420), pavurutamab (AMG 701), and teclistamab (Tecvayli®).

Accordingly, in these embodiments, the radiolabeled tracer is designed to bind the extracellular binding region of the BCMA-targeted CAR. In some embodiments, the ectodomain of the radiolabeled tracer has a BCMA-binding portion of the amino acid sequence provided at NCBI Accession No. NP_001183, incorporated herein by reference. In some embodiments, the ectodomain of the radiolabeled tracer has the following amino acid sequence:

(SEQ ID NO: 1)
MLQMAGQCSQNEYFDSLLHACIPCQLRCSSNTPPLTCQRYCNASVTNS
VKGTNA.

In some embodiments, the ectodomain of the radiolabeled tracer contains two repetitions of the extracellular domain of BCMA. The amino acid sequence of such a BCMA extracellular domain is set forth below (SEQ ID NO: 2):

| | |
|---|---|
| 1 | mlqmagqcsq neyfdsllha cipcqlrcss ntppltcqry cnasvtnsvk gtnagggsgg |
| 61 | gsprgsgggs mlqmagqcsq neyfdsllha cipcqlrcss ntppltcqry cnasvtnsvk |
| 121 | gtn |

In some embodiments, the subject was previously administered CAR immune cells that target cluster of differentiation antigen (CD) 19. CAR immune cells that target CD19 are known in the art. See, e.g., FDA-approved CAR-expressing immune cells lisocabtagene maraleucel (Breyanzi®), tisagenlecleucel (Kymriah®), brexucabtagene autoleucel (Tecartus®), and axicabtagene ciloleucel (Yescarta®), U.S. Pat. Nos. 9,629,877, 10,273,300, and 10,533,055, and U.S. Patent Application Publication Nos. 2020/0392248, and 2021/0238253. Such CAR immune cells may include an extracellular domain obtainable from a commercially available anti-CD19 antibody, anti-CD19⁻ binding fragment, or derivative thereof, e.g., loncastuximab (Zynlonta®), tafasitamab (Monjuvi®), denintuzumab (SGN-CD19A), and inebilizumab (Uplizna®).

Accordingly, in these embodiments, the radiolabeled tracer is designed to bind the extracellular binding region of the CD19⁻ targeted CAR. In some embodiments, the ectodomain of the radiolabeled tracer has a CD19⁻ binding portion of any one of the amino acid sequences provided at NCBI Accession Nos. XP_011544283, NP_001171569, NP_001372661, and NP_001761, incorporated herein by reference. In some embodiments, the CD19 ectodomain of the radiolabeled tracer has the amino acid sequence of any one of SEQ ID NOs: 3-5.

In some embodiments, the ectodomain of the radiolabeled tracer contains a variant of the extracellular domain of CD19, which is the approximately 32 kilodalton (kDa) portion of the CD19 protein (a total of approximately 58 kDa) to which anti-CD19 CAR molecules bind. The amino acid sequence of a representative variant of CD19 extracellular domain is set forth below SEQ ID NO: 3):

| | |
|---|---|
| 1 | peeplvvkve egdeawlpcl kgtsdgptqq ltwsresplk pflkvsfgvp glgvhvrpna |
| 61 | vslvisnvsq qmggfylcqp gppsekawqp gwtvnvegsg elfrwnvsdl gglgcglknr |
| 121 | ssegpsspsg klmspklyvw akdrpeiweg eppclpprds lnqslsrdmt vapgstlwls |
| 181 | cgvppdsvsr gplswthvhp kgpksllsle lkddrpardm wvtgtrlflp rataqdagky |
| 241 | ychrgnltms fhlevkarpv sahtklrtgg wk |

In some embodiments, the ectodomain has at least 85% sequence identity to SEQ ID NO: 3, at least 90% sequence identity to SEQ ID NO: 3, at least 95% sequence identity to SEQ ID NO: 3, at least 98% sequence identity to SEQ ID NO: 3, at least 99% sequence identity to SEQ ID NO: 3.

The amino acid sequence of a second representative variant of a CD19 extracellular domain is set forth below (SEQ ID NO: 4):

| | |
|---|---|
| 1 | peeplvvkve egdeawlpcl kgtsdgptqq ltwsresplk pflkvsfgvp glgvhvrpna |
| 61 | vslvisqvsq qmggfylcqp gppsekawqp gwtvnvegsg elfrwqvsdl gglgcglkqr |
| 121 | ssegpsspsg klmspklyvw akdrpeiweg eppclpprds lqqslsrdmt vapgstlwls |
| 181 | cgvppdsvsr gplswthvhp kgpksllsle lkddrpardm wvtgtrlflp rataqdagky |
| 241 | ychrgqltms fhlevkarpv sahtklrtgg wk |

The amino acid sequence of a third representative variant of a CD19 extracellular domain containing at the C-terminus thereof, a FLAG tag (DYKDDDDK (SEQ ID NO: 43), a sortase recognition motif (LPETG (SEQ ID NO: 47)), and a His6 tag (HHHHHH (SEQ ID NO: 42)), is set forth below (SEQ ID NO: 48):

| | |
|---|---|
| 1 | gspeeplvvk veegdeawlp clkgtsdgpt qqltwsresp lkpflkvsfg vpglgvhvrp |
| 61 | navslvisnv sqqmggfylc qpgppsekaw qpgwtvnveg sgelfrwnvs dlgglgcglk |
| 121 | nrssegpssp sgklmspkly vwakdrpeiw egeppclppr dslnqslsrd mtvapgstlw |
| 181 | lscgvppdsv srgplswthv hpkgpkslls lelkddrpar dmwvtgtrlf lprataqdag |
| 241 | kyychrgnlt msfhlevkar pvsahtklrt ggwkgsdykd dddklpetgh hhhhh |

The amino acid sequence of a representative CD19 extracellular domain set forth below (SEQ ID NO: 5):

| | |
|---|---|
| 1 | peeplvvkve egdnavlqcl kgtsdgptqq ltwsresplk pflklslglp glgihmrpla |
| 61 | iwlfifnvsq qmggfylcqp gppsekawqp gwtvnvegsg elfrwnvsdl gglgcglknr |
| 121 | ssegpsspsg klmspklyvw akdrpeiweg eppclpprds lnqslsqdlt mapgstlwls |
| 181 | cgvppdsvsr gplswthvhp kgpksllsle lkddrpardm wvmetglllp rataqdagky |
| 241 | ychrgnltms fhleitarpv lwhwllrtgg wk |

In some embodiments, the subject was previously administered CAR immune cells that target CD20. CAR extracellular domains and antibodies and fragments thereof that bind to CD20 are known in the art. See, e.g., U.S. Pat. Nos. 10,189,903, 10,442,867, 10,934,363, 11,066,457, 11,160,833, and 11,439,665, and U.S. Patent Application Publication No. 2018/0187149. In some embodiments, the CAR extracellular domain is obtainable from a commercially available anti-CD20 antibody, anti-CD20-binding fragment, or derivatives thereof, e.g., ofatumumab (Arzerra®, Kesimpta®), veltuzumab (IMMU-106), tositumomab (Bexxar®), and rituximab (Rituxan®, Riabni®, Truximab®).

Accordingly, in these embodiments, the radiolabeled tracer is designed to bind the extracellular binding region of the CD20-targeted CAR. In some embodiments, the ectodomain of the radiolabeled tracer has a CD20-binding portion of any one of the amino acid sequences provided at NCBI Accession Nos. NP_068769, NP_690605, and NP_690606, incorporated herein by reference. In some embodiments, the ectodomain of the radiolabeled tracer has the following amino acid sequence KISHFLKMESLNFIRAHTPYINIYNCEPANPSEKNSPSTQYCYSIQS (SEQ ID NO: 6).

In some embodiments, the subject was previously administered CAR immune cells that target CD22. CAR extracellular domains and antibodies and fragments thereof that bind to CD22 are known in the art. See, e.g., U.S. Pat. Nos. 9,139,649, 9,181,343, and 10,494,435, U.S. Patent Application Publication Nos. 2015/0175711, 2018/0086843, 2021/0047402, 2021/0095022, 2022/0220198, and 2022/0273710, and Fry et al., Nat. Med. 24(1):20-28 (2018). In some embodiments, the CAR extracellular domain is obtainable from a commercially available anti-CD22 antibody, anti-CD22-binding fragment, or derivatives thereof, e.g., bectumomab, epratuzumab, inotuzumab, moxetumomab, and epratuzumab.

Accordingly, in these embodiments, the radiolabeled tracer is designed to bind the extracellular binding region of the CD22-targeted CAR. In some embodiments, the ectodomain of the radiolabeled tracer has a CD22-binding portion of any one of the amino acid sequences provided at NCBI Accession Nos. NP_001172028, NP_001172029, NP_001172030, NP_001265346, and NP_001762, incorporated herein by reference. In some embodiments, the ectodomain of a radiolabeled tracer contains the extracellular domain of CD22. The amino acid sequence of a representative CD22 extracellular domain is set forth below (SEQ ID NO: 7):

| | |
|---|---|
| 1 | dsskwvfehp etlyawegac vwipctyral dgdlesfilf hnpeynknts kfdgtrlyes |
| 61 | tkdgkvpseq krvqflgdkn knctlsihpv hlndsgqlgl rmesktekwm erihlnvser |
| 121 | pfpphiqlpp eiqesqevtl tcllnfscyg ypiqlqwlle gvpmrqaavt stsltiksvf |
| 181 | trselkfspq wshhgkivtc qlqdadgkfl sndtvqlnvk htpkleikvt psdaivregd |
| 241 | svtmtcevss snpeyttvsw lkdgtslkkq ntftlnlrev tkdqsgkycc qvsndvgpgr |
| 301 | seevflqvqy apepstvqil hspavegsqv eflcmslanp lptnytwyhn gkemqgrtee |
| 361 | kvhipkilpw hagtyscvae nilgtgqrgp gaeldvqypp kkvttviqnp mpiregdtvt |
| 421 | lscnynssnp svtryewkph gaweepslgv lkiqnvgwdn ttiacaacns wcswaspval |
| 481 | nvqyaprdvr vrkikplsei hsgnsvslqc dfssshpkev qffwekngrl lgkesqlnfd |
| 541 | sispedagsy scwvnnsigq taskawtlev lyaprrlrvs mspgdqvmeg ksatltcesd |
| 601 | anppvshytw fdwnnqslpy hsqklrlepv kvqhsgaywc qgtnsvgkgr splstltvyy |
| 661 | spetigrr |

In some embodiments, the subject was previously administered CAR immune cells that target signaling lymphocyte activation marker family member (SLAMF) 7. CAR extracellular domains that bind SLAMF7 are known in the art. See, e.g., U.S. Pat. No. 10,799,536, and U.S. Patent Application Publication Nos. 2020/0024342, 2020/0283534, 2021/0230548, and 2021/0253729. In some embodiments, the CAR extracellular domain is obtainable from a commercially available anti-SLAMF7 antibody, anti-SLAMF7-binding fragment, or derivative thereof, e.g., elotuzumab (Empliciti®).

Accordingly, in these embodiments, the radiolabeled tracer is designed to bind the extracellular binding region of the SLAMF7-targeted CAR. In some embodiments, the ectodomain of the radiolabeled tracer has a SLAMF7-binding portion of any one of the amino acid sequences provided at NCBI Accession Nos. XP_011508130, XP_011508131, XP_047282315, NP_001269517, NP_001269518, NP_001269519, NP_001269520, NP_001269521, NP_001269522, NP_001269523, NP 001269524, NP_001269525, and NP_067004, incorporated herein by reference. In some embodiments, the ectodomain of the radiolabeled tracer contains an extracellular domain of SLAMF7. The amino acid sequence of a representative SLAMF7 extracellular domain is set forth below (SEQ ID NO: 8):

| | |
|---|---|
| 1 | sgpvkelvgs vggavtfplk skvkqvdsiv wtfnttplvt iqpeggtiiv tqnrnrervd |
| 61 | fpdggyslkl sklkkndsgi yyvgiysssl qqpstqeyvl hvyehlskpk vtmglqsnkn |
| 121 | gtcvtnltcc mehgeedviy twkalgqaan eshngsilpi swrwgesdmt ficvarnpvs |
| 181 | rnfsspilar klcegaaddp dssm |

In some embodiments, the subject was previously administered CAR immune cells that target programmed cell death 1 (PD-1). CAR extracellular domains that bind PD-1 are known in the art. See, e.g., U.S. Pat. Nos. 10,124,023 and 11,136,392, and U.S. Patent Application Publication Nos. 2021/0061877, 2020/0281974, and 2022/0064595. In some embodiments, the CAR extracellular domain is obtainable from a commercially available anti-PD-1 antibody, anti-PD-1-binding fragment, or derivative thereof, e.g., balstilimab, budigalimab, cadonilimab, cemiplimab (Libtayo®), cetrelimab, dostarlimab (Jemperli®), izuralimab, nivolumab (Opdivo®), pacmilimab, pembrolizumab (Keytruda®), penpulimab, peresolimab, pidilizumab, retifanlimab, rosnilimab, sintilimab, spartalizumab, tislelizumab, toripalimab, volrustomig, vudalimab, zeluvalimab, and zimberelimab.

Accordingly, in these embodiments, the radiolabeled tracer is designed to bind the extracellular binding region of the PD-I-targeted CAR. In some embodiments, the ectodomain of the radiolabeled tracer has a PD-1-binding portion of any one of the amino acid sequences provided at NCBI Accession Nos. XP_006712636, and NP_005009, incorporated herein by reference. In some embodiments, the ectodomain of the radiolabeled tracer contains an extracellular domain of PD-1. The amino acid sequence of a representative PD-1 extracellular domain is set forth below (SEQ ID NO: 9):

| | |
|---|---|
| 1 | fldspdrpwn pptfspallv vtegdnatft csfsntsesf vlnwyrmsps nqtdklaafp |
| 61 | edrsqpgqdc rfrvtqlpng rdfhmsvvra rrndsgtylc gaislapkaq ikeslraelr |
| 121 | vterraevpt ahpspsprpa gqfqtlv |

In some embodiments, the subject was previously administered CAR immune cells that target KIT Proto-Oncogene, Receptor Tyrosine Kinase (KIT). CAR extracellular domains, and antibodies and fragments thereof that bind to KIT are known in the art. See, e.g., U.S. Patent Application Publication Nos. 2017/0335281, 2020/0048359, 2020/0071397, and 2021/0299177. In some embodiments, the CAR extracellular domain is obtainable from a commercially available anti-KIT antibody, anti-KIT-binding fragment, or derivative thereof, e.g., barzolvolimab.

Accordingly, in these embodiments, the radiolabeled tracer is designed to bind the extracellular binding region of the KIT-targeted CAR. In some embodiments, the ectodomain of the radiolabeled tracer has a KIT-binding portion of any one of the amino acid sequences provided at NCBI Accession Nos. NP_000213, NP_001087241, NP_001372213, NP_001372214, NP_001372215, NP_001372217, NP_001372219, and NP_001372221, incorporated herein by reference. In some embodiments, the ectodomain of the radiolabeled tracer contains an extracellular domain of KIT. The amino acid sequence of a representative KIT extracellular domain is set forth below (SEQ ID NO: 10):

| | |
|---|---|
| 1 | qpsvspgeps ppsihpgksd livrvgdeir llctdpgfvk wtfeildetn enkqnewite |
| 61 | kaeatntgky tctnkhglsn siyvfvrdpa klflvdrsly gkedndtlvr cpltdpevtn |
| 121 | yslkgcqgkp lpkdlrfipd pkagimiksv krayhrlclh csvdqegksv lsekfilkvr |
| 181 | pafkavpvvs vskasyllre geeftvtcti kdvsssvyst wkrensqtkl qekynswhhg |
| 241 | dfnyerqatl tissarvnds gvfmcyannt fgsanvtttl evvdkgfini fpminttvfv |
| 301 | ndgenvdliv eyeafpkpeh qqwiymnrtf tdkwedypks enesniryvs elhltrlkgt |
| 361 | eggtytflvs nsdvnaaiaf nvyvntkpei ltydrlvngm lqcvaagfpe ptidwyfcpg |
| 421 | teqrcsasvl pvdvqtlnss gppfgklvvq ssidssafkh ngtveckayn dvgktsayfn |
| 481 | fafkgnnkeq ihphtlftp |

In some embodiments, the subject was previously administered CAR immune cells that target CD38. CAR extracellular domains and antibodies and fragments thereof that bind to CD38 are known in the art. See, e.g., U.S. Pat. Nos. 10,709,775, 10,799,536, 10,836,998, and 11,365,394, and U.S. Patent Application Publication Nos. 2017/0296623, 2019/0135894, 2019/0135937, 2020/0308541, 2021/0046118, and 2022/0202859. In some embodiments, the CAR extracellular domain is obtainable from a commercially available anti-CD38 antibody, anti-CD38-binding fragment, or derivative thereof, e.g., daratumumab (Darzalex®), isatuximab (Sarclisa®), and mezagitamab.

Accordingly, in these embodiments, the radiolabeled tracer is designed to bind the extracellular binding region of the CD38-targeted CAR. In some embodiments, the ectodomain of the radiolabeled tracer has a CD38-binding portion of the amino acid sequence provided at NCBI Accession No. 001766, incorporated herein by reference. In some embodiments, the ectodomain of the radiolabeled tracer contains an extracellular domain of CD38. The amino acid sequence of a representative CD38 extracellular domain is set forth below (SEQ ID NO: 11):

| | |
|---|---|
| 1 | vprwrqqwsg pgttkrfpet vlarcvkyte ihpemrhvdc qsvwdafkga fiskhpcnit |
| 61 | eedyqplmkl gtqtvpcnki llwsrikdla hqftqvqrdm ftledtllgy laddltwcge |
| 121 | fntskinyqs cpdwrkdcsn npvsvfwktv srrfaeaacd vvhvmlngsr skifdknstf |
| 181 | gsvevhnlqp ekvqtleawv ihggredsrd lcqdptikel esiiskrniq fsckniyrpd |
| 241 | kflqcvknpe dssctsei |

In some embodiments, the subject was previously administered CAR immune cells that target Claudin 18.2. CAR extracellular domains and antibodies and fragments thereof that bind to Claudin 18.2 are known in the art. Accordingly, in these embodiments, radiolabeled tracer is designed to bind the extracellular binding region of the Claudin 18.2-targeted CAR. In some embodiments, the ectodomain of the radiolabeled tracer has a Claudin 18.2-binding portion of the amino acid sequence provided at NCBI Accession No. NP_001002026, incorporated herein by reference. In some embodiments, the ectodomain of the radiolabeled tracer contains an extracellular domain of Claudin 18.2. The amino acid sequence of a representative Claudin 18.2 first extracellular domain is set forth below (SEQ ID NO: 12):

| | |
|---|---|
| 1 | dqwstqdlyn npvtavfnyq glwrscvres sgftecrgyf tllglpamlq avr |

The amino acid sequence of a representative Claudin 18.2 second extracellular domain is set forth below (SEQ ID NO: 13):

| | |
|---|---|
| 1 | vtnfwmstan mytgmggmvq tvqtrytfga a |

In some embodiments, the subject was previously administered CAR immune cells that target Tumor associated calcium signal transducer 2 (TACSTD2; TROP2). CAR extracellular domains and antibodies and fragments thereof that bind to TROP2 are known in the art. Accordingly, in these embodiments, radiolabeled tracer is designed to bind the extracellular binding region of the TROP2-targeted CAR. In some embodiments, the ectodomain of the radiolabeled tracer has a TROP-binding portion of the amino acid sequence provided at NCBI Accession No. NP_002344, incorporated herein by reference. In some embodiments, the ectodomain of the radiolabeled tracer contains an extracellular domain of TROP2. The amino acid sequence of a representative TROP2 extracellular domain is set forth below (SEQ ID NO: 14):

| | |
|---|---|
| 1 | htaaqdnctc ptnkmtvcsp dgpggrcqcr algsgmavdc stltskclll karmsapkna |
| 61 | rtlvrpseha lvdndglydp dcdpegrfka rqcnqtsvcw cvnsvgvrrt dkgdlslrcd |
| 121 | elvrthhili dlrhrptaga fnhsdldael rrlfreryrl hpkfvaavhy eqptiqielr |
| 181 | qntsqkaagd vdigdaayyf erdikgeslf qgrggldlrv rgeplqvert liyyldeipp |
| 241 | kfsmkrlt |

In some embodiments, the subject was previously administered CAR immune cells that target mesothelin (MSLN). CAR extracellular domains and antibodies and fragments thereof that bind to MSLN are known in the art. Accordingly, in these embodiments, radiolabeled tracer is designed to bind the extracellular binding region of the MSLN-targeted CAR. In some embodiments, the ectodomain of the radiolabeled tracer has a MSLN-binding portion of any one of the amino acid sequences provided at NCBI Accession Nos. NP_001170826, NP_005814, and NP_037536, incorporated herein by reference. In some embodiments, the ectodomain of the radiolabeled tracer is derived from MSLN. MSLN is a GPI-anchored protein, therefore the entire MSLN protein is extracellular. The amino acid sequence of a representative MSLN is set forth below (SEQ ID NO: 15):

| | |
|---|---|
| 1 | malptarpll gscgtpalgs llfllfslgw vqpsrtlage tgqeaapldg vlanppniss |
| 61 | lsprqllgfp caevsglste rvrelavala qknvklsteq lrclahrlse ppedldalpl |
| 121 | dlllflnpda fsgpqactrf fsritkanvd llprgaperq rllpaalacw gvrgsllsea |
| 181 | dvralgglac dlpgrfvaes aevllprlvs cpgpldqdqq eaaraalqgg gppygppstw |
| 241 | svstmdalrg llpvlgqpii rsipqgivaa wrqrssrdps wrqpertilr prfrrevekt |
| 301 | acpsgkkare ideslifykk weleacvdaa llatqmdrvn aipftyeqld vlkhkldely |
| 361 | pqgypesviq hlgylflkms pedirkwnvt sletlkalle vnkghemspq aprrplpqva |
| 421 | tlidrfvkgr gqldkdtldt ltafypgylc slspeelssv ppssiwavrp qdldtcdprq |
| 481 | ldvlypkarl afqnmngsey fvkiqsflgg aptedlkals qqnvsmdlat fmklrtdavl |
| 541 | pltvaevqkl lgphveglka eerhrpvrdw ilrqrqddld tlglglqggi pngylvldls |
| 601 | mqealsgtpc llgpgpvltv lalllastla |

In some embodiments, the ectodomain of the radiolabeled tracer contains a portion of the MSLN protein. In some embodiments, the ectodomain of the radiolabeled tracer is IPNGYLVLDLSMQEALS (SEQ ID NO: 16). In some embodiments, the ectodomain of the radiolabeled tracer is YNVNDLSMQEL (SEQ ID NO: 17), where N is any amino acid.

The amino acid sequences of additional representative cancer antigens that may be targeted by CAR immune cells, and from which an ectodomain may be determined or obtained from are provided at the NCBI Accession numbers set forth in Table 1, and are incorporated herein by reference.

TABLE 1
Gene Name, Symbols, and NCBI Accession Numbers of Representative Cancer Antigens

| Gene Name | Gene Symbols | Protein Accession No(s). |
|---|---|---|
| Alpha fetoprotein | AFP, HPAFP, FETA, AFPD | NP_001125, NP_001341646 |
| Alkaline phosphatase, | ALP, ALPI, ALPP, IAP, | NP_001623 |
| placental | PALP, PLAP, PLAP-1 | |
| Axl receptor tyrosine | AXL, UFO, JTK11, Tyro7, | NP_001265528, NP_001690, |
| kinase | ARK | NP_068713 |
| Beta-1,4-n-acetyl- | B4GALNT1, GALGT, 48, | XP_005268830, XP_011536449, |
| galactosaminyltransferase | GalNAc-T, SPG26, | XP_016874629, XP_016874631, |
| 1 | GALNACT, SIAT2 | XP_024304696, XP_047284636, |
| | | XP_047284637, XP_047284638, |
| | | NP_001263397, NP_001263398, |
| | | NP_001469 |
| Tumor necrosis factor | TNFRSF17, BCM, BCMA, | NP_001183.2 |
| receptor superfamily | CD269 | |
| member 17 | | |
| Carbonic anhydrase 9 | CA9, CAIX, MN, P54/58N, | XP_047279805, XP_047279806, |
| | CA-IX, PMW1, G250 | NP_001207 |
| CD5 molecule | CD5, LEU1, T1 | NP_001333385, NP_055022 |
| CD7 molecule | CD7, GP40, TP41, LEU-9, | NP_006128 |
| | Tp40 | |
| CD20 molecule | See Membrane spanning 4- | |
| | domains A1 entry below | |
| CD22 molecule | CD22, SIGLEC2, SIGLEC-2, | NP_001172028, NP_001172029, |
| | BL-CAM, Siglec-2 | NP_001172030, NP_001265346, |
| | | NP_001762 |
| Fc epsilon receptor II | FCER2, CLEC4J, FCErII, | XP_005272519, NP_001193948, |
| | CD23A, CD23, FCE2, | NP_001207429, NP_001993 |
| | BLAST-2, IGEBF | |
| CD33 molecule | CD33, SIGLEC3, SIGLEC-3, | XP_011525833, XP_011525834, |
| | P67, FLJ00391, Gp67, | XP_016882997, XP_016882998, |
| | Siglec-3 | XP_016882999, XP_047295684, |
| | | XP_047295685, XP_047295686, |
| | | XP_047295687, XP_047295688, |
| | | NP_001076087, NP_001171079, |
| | | NP_001763 |
| CD34 molecule | CD34 | NP_001020280, NP_001764 |
| CD44 molecule | CD44, HUTCH-I, HCELL, | XP_005253288, XP_005253289, |
| | CSPG8, MC56, Pgp1, MDU2, | XP_005253292, XP_005253295, |
| | MDU3, MIC4, IN, Hermes-1, | XP_005253296, XP_005253297, |
| | ECMR-III, HUTCH-1, ECM- | XP_006718451, XP_006718453, |
| | III, Epican, PGP-1 CD44R, | XP_011518784, XP_011518785, |
| | CDw44, H-CAM, LHR, | XP_011518786, XP_011518787, |
| | CDW44, PGP-I | XP_011518788, XP_011518789, |
| | | XP_011518790, XP_011518791, |
| | | XP_016874072, XP_047283851, |
| | | XP_047283852, XP_047283853, |
| | | XP_047283854, XP_047283855, |
| | | XP_047283856, XP_047283857, |
| | | XP_047283858, XP_047283859, |
| | | XP_047283860, NP_000601, |
| | | NP_001001389, NP_001001390, |
| | | NP_001001391, NP_001001392, |
| | | NP_001189484, NP_001189485, |
| | | NP_001189486 |
| CD52 molecule | CD52, HE5, EDDM5, | NP_001794 |
| | CDW52, CDw52, He5 | |
| CD70 molecule | CD70, CD27L, CD27-L, | NP_001243, NP_001317261 |
| | CD27LG, TNFSF7, TNLG8A, | |
| | LPFS3 | |
| CD80 molecule | B7, B7-1, B7.1, BB1, | NP_005182 |
| | CD28LG, CD28LG1, CD80, | |
| | LAB7 | |
| CD86 molecule | B7-2, B7.2, B70, BU63, | NP_001193853, NP_001193854, |
| | CD28LG2, CD86, FUN-1, | NP_008820, NP_787058, |
| | LAB72 | NP_795711 |
| Basigin | 5F7, BSG, Basigin, CD147, | NP_001309172, NP_001719, |
| | EMMPRIN, EMPRIN, | NP_940991, NP_940992, |
| | HAb18G, OK, TCSF | NP_940993 |
| Interleukin 3 receptor | CD123, HIL-3Ra, IL-3RA, | XP_005274488, XP_005274489, |
| subunit alpha | IL3R, IL3RA, IL3RAY, | XP_005274837, XP_005274838, |
| | IL3RX, IL3RY | XP_016884980, XP_016885532, |
| | | XP_047298046, XP_047298047, |
| | | XP_047298686, XP_047298687, |
| | | NP_001254642, NP_002174 |
| Prominin 1 | AC133, CD133, CORD12, | XP_005248252, XP_005248253, |
| | Hprominin, MCDR2, | XP_006714037, XP_011512195, |
| | MSTP061, PROM1, | XP_011512196, XP_011512197, |
| | PROML1, RP41, STGD4 | XP_011512199, XP_011512202, |
| | | XP_011512204, XP_011512205, |
| | | XP_016864289, XP_047272326, |
| | | XP_047272328, XP_047272329, |
| | | XP_047272330, XP_047272331, |
| | | XP_047272332, XP_047272333, |
| | | XP_047272334, XP_047272335, |
| | | NP_001139319, NP_001139320, |
| | | NP_001139321, NP_001139322, |
| | | NP_001139323, NP_001139324, |
| | | NP_001358335, NP_001358336, |
| | | NP_001358337, NP_006008 |
| Fucosyltransferase 3 | CD174, FT3B, FUT3, FucT- | NP_000140, NP_001091108, |
| (lewis blood group) | III, LE, Les, Lewis FT | NP_001091109, NP_001091110, |
| | | NP_001361669, NP_001369673, |
| | | NP_001369674, NP_001369675, |
| | | NP_001369676, NP_001369677, |
| | | NP_001369678, NP_001369679 |
| CD274 molecule | B7-H, B7-H1, B7H1, CD274, | XP_047279218, NP_001254635, |
| | HPD-L1, PD-L1, PDCD1L1, | NP_001300958, NP_054862 |
| | PDCD1LG1, PDL1 | |
| CD276 molecule | 4Ig-B7-H3, B7-H3, B7H3, | XP_005254757, XP_011520397, |
| | B7RP-2, CD276 | XP_016878127, XP_047289103, |
| | | XP_047289104, NP_001019907, |
| | | NP_001316557, NP_001316558, |
| | | NP_079516 |
| CD248 molecule | CD164L1, CD248, TEM1 | NP_065137 |
| carcinoembryonic antigen | CD66e, CEA, CEACAM5 | XP_011524624, XP_016881634, |
| (CEA) cell adhesion | | XP_016881635, NP_001278413, |
| molecule 5 | | NP_001295327, NP_004354 |
| C-type lectin domain | CD371, CLEC12A, CLL-1, | XP_005253381, XP_006719096, |
| family 12 member A | CLL1, DCAL-2, DCAL2, | XP_006719098, XP_006719099, |
| | MICL | XP_011518872, XP_011518873, |
| | | XP_011518875, XP_047284353, |
| | | XP_047284355, XP_047284356, |
| | | XP_047284357, XP_047284358, |
| | | NP_001193939, NP_001287659, |
| | | NP_612210, NP_963917 |
| Claudin 1 | CLD1, CLDN1, Claudin 1, | NP_066924 |
| | Claudin-1, ILVASC, SEMP1 | |
| Claudin 6 | CLDN6, Claudin 6, Claudin-6, | NP_067018 |
| | Skullin | |
| Claudin 18 | CLDN18, SFTA5, SFTPJ | NP_057453 |
| Cytotoxic T-lymphocyte | ALPS5, CD, CD152, | NP_001032720, NP_005205 |
| associated protein 4 | CELIAC3, CTLA-4, CTLA4, | |
| | GRD4, GSE, IDDM12 | |
| Cancer/testis antigen 1B | CT6.1, CTAG, CTAG1, | NP_001318 |
| | CTAG1B, ESO1, LAGE-2, | |
| | LAGE2, LAGE2A, LAGE2B, | |
| | NY-ESO-1 | |
| Chondroitin sulfate | CSPG4, CSPG4A, HMW- | XP_047288152, NP_001888 |
| proteoglycan 4 | MAA, MCSP, MCSPG, MEL- | |
| | CSPG, MSK16, NG2 | |
| Delta like canonical notch | DLL3, Delta3, SCDO1 | NP_058637, NP_982353 |
| ligand 3 | | |
| Epidermal growth factor | EGFR, EGFR VIII, ERBB, | XP_047275908, XP_047275909, |
| receptor | ERBB1, ERRP, HER1, | NP_001333826, NP_001333827, |
| | MENA, NISBD2, PIG61 | NP_001333828, NP_001333829, |
| | | NP_001333870, NP_005219, |
| | | NP_958439, NP_958440, |
| | | NP_958441 |
| Epithelial cell adhesion | Ber-Ep4, BerEp4, CD326, | NP_002345 |
| molecule | CO-17A, DIAR5, EGP, EGP- | |
| | 2, EGP314, EGP34, EGP40, | |
| | EPCAM, ESA, Ep-CAM, | |
| | GA733-2, HEA125, | |
| | HEGP314, HNPCC8, KS1/4, | |
| | KSA, Ly74, M1S2, M4S1, | |
| | MH99, MIC18, MK-1, MOC- | |
| | 31, MOC31, TACST-1, | |
| | TACSTD1, TROP1 | |
| EPH receptor A2 | ARCC2, CTPA, CTPP1, | XP_016856026, XP_047304215, |
| | CTRCT6, EC 2.7.10, ECK, | XP_047304223, XP_047304228, |
| | EPHA2, EphA2 | NP_001316019, NP_004422 |
| Erb-B2 receptor tyrosine | C-ERB-2, C-ERB2, CD340, | XP_047291546, NP_001005862, |
| kinase 2 | ERBB2, HER-2, HER-2/Neu, | NP_001276865, NP_001276866, |
| | HER2, Herstatin, MLN 19, | NP_001276867, NP_001369711, |
| | MLN-19, MLN19, NEU, | NP_001369712, NP_001369713, |
| | NGL, P185erbB2, TKR1, | NP_001369714, NP_001369715, |
| | VSCN2 | NP_001369716, NP_001369717, |
| | | NP_001369718, NP_001369719, |
| | | NP_001369720, NP_001369721, |
| | | NP_001369722, NP_001369723, |
| | | NP_001369724, NP_001369725, |
| | | NP_001369726, NP_001369727, |
| | | NP_001369728, NP_001369729, |
| | | NP_001369730, NP_001369731, |
| | | NP_001369732, NP_001369733, |
| | | NP_001369734, NP_001369735, |
| | | NP_004439 |
| Fibroblast activation | DPPIV, FAP, FAPA, FAPα, | XP_011509098, XP_011509099, |
| protein α | SIMP, Seprase | XP_016859074, NP_001278736, |
| | | NP_004451 |
| Folate hydrolase 1 | FGCP, FOLH, FOLH1, GCP2, | XP_011518260, XP_016872921, |
| | GCPII, MGCP, NAALAD1, | XP_016872922, XP_016872923, |
| | NAALAdase, PSM, PSMA | XP_047282634, XP_047282635, |
| | | XP_047282636, XP_047282637, |
| | | XP_047282638, NP_001014986, |
| | | NP_001180400, NP_001180401, |
| | | NP_001180402, NP_001338165, |
| | | NP_004467 |
| Folate receptor α | FBP, FOLR, FOLR1, FR-α, | NP_000793, NP_057936, |
| | FRα, FRα, NCFTD | NP_057937, NP_057941 |
| Glypican 3 | DGSX, GPC3, GTR2-2, | XP_016884902, NP_001158089, |
| | MXR7, OCI-5, OCI5, SDYS, | NP_001158090, NP_001158091, |
| | SGB, SGBS, SGBS1 | NP_004475 |
| G protein-coupled | GPRC5D | XP_016875072, NP_061124 |
| receptor class C group 5 | | |
| member D | | |
| Glycoprotein Nmb | GPNMB, HGFIN, NMB, | XP_005249635, XP_016867167, |
| | PLCA3 | XP_047275732, NP_001005340, |
| | | NP_002501 |
| Human papillomavirus | E7 | NP_041326 |
| transforming early protein | | |
| 7 | | |
| Interleukin 1 receptor | C3orf13, IL-1R-3, IL-1R3, | XP_016861837, XP_047304037, |
| accessory protein | IL-1RacP, IL1R3, IL1RAP | XP_047304038, XP_047304039, |
| | | XP_047304040, XP_047304041, |
| | | XP_047304042, NP_001161400, |
| | | NP_001161401, NP_001161402, |
| | | NP_001161403, NP_001351808, |
| | | NP_001351809, NP_001351810, |
| | | NP_002173, NP_608273 |
| Interleukin 3 receptor | CD123, HIL-3Rα, IL-3RA, | XP_005274488, XP_005274489, |
| subunit α | IL3R, IL3RA, IL3RAY, | XP_005274837, XP_005274838, |
| | IL3RX, IL3RY | XP_016884980, XP_016885532, |
| | | XP_047298046, XP_047298047, |
| | | XP_047298686, XP_047298687, |
| | | NP_001254642, NP_002174 |
| Interleukin 6 receptor | CD126, Gp80, HIES5, IL-1Ra, | XP_005245196, XP_016856688, |
| | IL-6R, IL-6R 1, IL-6R-1, IL- | XP_047275604, XP_047275605, |
| | 6RA, IL6Q, IL6QTL, IL6R, | XP_047275606, XP_047275610, |
| | IL6RA, IL6RQ | XP_047275611, XP_047275612, |
| | | XP_047275613, NP_000556, |
| | | NP_001193795, NP_001369698, |
| | | NP_001369699, NP_001369700, |
| | | NP_001369701, NP_001369702, |
| | | NP_001369703, NP_852004 |
| Interleukin 13 receptor | CD213A2, CD213α2, CT19, | NP_000631 |
| subunit α 2 | IL-13R, IL-13RA2, IL13BP, | |
| | IL13R, IL13RA2 | |
| Kinase insert domain | CD309, FLK-1, FLK1, KDR, | NP_002244 |
| receptor | VEGFR, VEGFR-2, VEGFR2 | |
| Killer cell lectin like | CD314, D12S2489E, KLR, | NP_031386 |
| receptor K1 | KLRK1, NKG2-D, NKG2D | |
| L1 cell adhesion molecule | CAML1, CD171, HSAS, | NP_000416, NP_001137435, |
| | HSAS1, L1CAM, MASA, | NP_001265045, NP_076493 |
| | MIC5, N-CAM-L1, N- | |
| | CAML1, NCAM-L1, S10, | |
| | SPG1 | |
| Mage family member A1 | CT1.1, MAGE1, MAGE1A, | NP_004979 |
| | MAGEA1, MGC9326 | |
| Mage family member A4 | CT1.4, MAGE-41, MAGE- | NP_001011548, NP_001011549, |
| | X2, MAGE4, MAGE4A, | NP_001011550, NP_001373125, |
| | MAGE4B, MAGEA4, | NP_001373126, NP_001373127, |
| | MGC21336 | NP_001373128, NP_001373129, |
| | | NP_001373131, NP_001373132, |
| | | NP_002353 |
| Met proto-oncogene, | AUTS9, C-Met, DA11, | XP_011514525, XP_047276356, |
| receptor tyrosine kinase | DFNB97, HGFR, MET, | NP_000236, NP_001120972, |
| | RCCP2 | NP_001311330, NP_001311331 |
| Membrane | CALLA, CD10, CMT2T, | XP_006713710, XP_011511158, |
| metalloendopeptidase | EPN, MME, NEP, SCA43, | XP_011511159, XP_047304113, |
| | SFE | NP_000893, NP_001341571, |
| | | NP_001341572, NP_001341573, |
| | | NP_009218, NP_009219, |
| | | NP_009220 |
| Mucin 1, cell surface | ADMCKD, ADMCKD1, | NP_001018016, NP_001018017, |
| associated | ADTKD2, CA 15-3, CD227, | NP_001037855, NP_001037856, |
| | Ca15-3, EMA, Episialin, | NP_001037857, NP_001037858, |
| | H23AG, KL-6, MAM6, MCD, | NP_001191214, NP_001191215, |
| | MCKD, MCKD1, MUC-1, | NP_001191216, NP_001191217, |
| | MUC-1/SEC, MUC-1/X, | NP_001191218, NP_001191219, |
| | MUC1, MUC1/ZD, Mucin-1, | NP_001191220, NP_001191221, |
| | PEM, PEMT, PUM | NP_001191222, NP_001191223, |
| | | NP_001191224, NP_001191225, |
| | | NP_001191226, NP_001358649, |
| | | NP_002447 |
| Mucin 16, cell surface | CA-125, CA125, FLJ14303, | NP_001388430, NP_078966 |
| associated | MUC-16, MUC16, Mucin-16 | |
| Neural cell adhesion | CD56, MSK39, N-CAM-1, | NP_000606, NP_001070150, |
| molecule 1 | NCAM, NCAM-1, NCAM1 | NP_001229536, NP_001229537, |
| | | NP_001373218, NP_001373219, |
| | | NP_001373220, NP_001373221, |
| | | NP_001387532, NP_001387533, |
| | | NP_001387534, NP_001387535, |
| | | NP_001387536, NP_001387537, |
| | | NP_001387538, NP_001387539, |
| | | NP_001387540, NP_001387541, |
| | | NP_001387542, NP_001387543, |
| | | NP_001387544, NP_001387545, |
| | | NP_001387546, NP_001387547, |
| | | NP_001387548, NP_001387549, |
| | | NP_001387550, NP_001387551, |
| | | NP_001387552, NP_001387553, |
| | | NP_851996 |
| Programmed cell death 1 | CD279, HPD-1, HPD-L, | XP_006712636, NP_005009 |
| | HSLE1, PD-1, PD1, PDCD1, | |
| | SLEB2 | |
| Premelanosome protein | D12S53E, Gp100, HMB-45, | NP_001186982, NP_001186983, |
| | HMB45, ME20, ME20-M, | NP_001307050, NP_001307051, |
| | ME20M, P1, P100, PMEL, | NP_001371290, NP_008859 |
| | PMEL17, Pmel17, SI, SIL, | |
| | SILV | |
| Prominin 1 | AC133, CD133, CORD12, | XP_005248252, XP_005248253, |
| | Hprominin, MCDR2, | XP_006714037, XP_011512195, |
| | MSTP061, PROM1, | XP_011512196, XP_011512197, |
| | PROML1, RP41, STGD4 | XP_011512199, XP_011512202, |
| | | XP_011512204, XP_011512205, |
| | | XP_016864289, XP_047272326, |
| | | XP_047272328, XP_047272329, |
| | | XP_047272330, XP_047272331, |
| | | XP_047272332, XP_047272333, |
| | | XP_047272334, XP_047272335, |
| | | NP_001139319, NP_001139320, |
| | | NP_001139321, NP_001139322, |
| | | NP_001139323, NP_001139324, |
| | | NP_001358335, NP_001358336, |
| | | NP_001358337, NP_006008 |
| Prostate stem cell antigen | LncPSCA, PRO232, PSCA | NP_005663 |
| Receptor tyrosine kinase | NTRKR1, ROR1 | XP_011539828, XP_016856865, |
| like orphan receptor 1 | | XP_016856866, NP_001077061, |
| | | NP_005003 |
| Receptor tyrosine kinase | BDB, BDB1, NTRKR2, | XP_005252065, XP_005252066, |
| like orphan receptor 2 | ROR2 | XP_006717184, XP_016870251, |
| | | XP_047279390, XP_047279391, |
| | | XP_047279392, XP_047279393, |
| | | NP_001305133, NP_004551 |
| Syndecan 1 | CD138, SDC, SDC1, SYND1 | XP_005262677, XP_005262678, |
| | | XP_005262679, NP_001006947, |
| | | NP_002988 |
| TNF receptor superfamily | CD30, DIS166E, KI-1, Ki-1, | XP_011540743, XP_011540745, |
| member 8 | TNFRSF8 | XP_047290749, XP_047290755, |
| | | NP_001234, NP_001268359 |
| TNF receptor superfamily | CD262, DR5, KILLER, | NP_003833, NP_671716 |
| member 10B | TNFRSF10B, TRAIL-R2, | |
| | TRAILR2, TRICK2, | |
| | TRICK2A, TRICK2B, | |
| | TRICKB, ZTNFR9 | |
| TNF receptor superfamily | BAFF-R, BAFFR, BR3, | NP_443177 |
| member 13C | BROMIX, CD268, CVID4, | |
| | Prolixin, TNFRSF13C | |
| Ubiquitin c-terminal | HEL-117, HEL-S-53, | NP_004172 |
| hydrolase L1 | NDGOA, PARK5, PGP 9.5, | |
| | PGP9.5, PGP95, SPG79, | |
| | UCH-L1, UCHL-1, UCHL1, | |
| | Uch-L1 | |
| UL16 binding protein 1 | N2DL-1, N2DL1, NKG2DL1, | XP_016866811, NP_001304018, |
| | RAET1I, ULBP1 | NP_079494 |
| UL16 binding protein 2 | N2DL-2, N2DL2, NKG2DL2, | XP_016866810, XP_047275333, |
| | RAET1H, RAET1L, ULBP2 | NP_079493 |

The ectodomains of the radiolabeled tracers are not limited to known cancer antigens. Unique cancer antigens (neoantigens) may be determined by known methods. For example, cancer genomes can be compared with normal cell genomes to identify neoantigens. In some embodiments, cancer transcriptomes are compared to normal cell transcriptomes. Computational methods may then be utilized to identify suitable binding sites for a CAR.

### Radioactive Isotopes

The radioactive isotope enables detection via PET or SPECT. In some embodiments, the radioactive isotope is suitable for detection in PET-computed tomography (PET-CT) or combined PET and magnetic resonance imaging (MI) (PET-MRI). The choice of the radioactive isotope may depend on factors such as half-life, availability, and ease of attachment to the tracer.

Representative examples of radioactive isotopes that may be contained in the radiolabeled tracer include Carbon-11 (¹¹C, ~20.4 m half-life), Nitrogen-13 (¹³N, ~10 m half-life), Oxygen-15 (¹⁵O, ~2 m half-life), Gallium-68 (⁶⁸Ga, ~68 m half-life), Fluorine-18 (¹⁸F, ~109.7 m half-life), Gallium-66 (⁶⁶Ga, ~9.5 h half-life), Copper-64 (⁶⁴Cu, ~12.7 h half-life), iodine-123 (¹²³I, ~13.2 h half-life), niobium-90 (⁹⁰Nb, ~14.5 h half-life), bromine-76 (⁷⁶Br, ~16.2 h half-life), Yttrium-86 (⁸⁶Y, ~17.4 h half-life), Cobalt-55 (¹¹Co, ~17.5 h half-life), Terbium-152 (¹⁵²Tb, ~17.5 h half-life), Arsenic-72 (⁷²As, ~20 h half-life), and Germanium-69 (⁶⁹Ge, ~39 h half-life), indium (¹¹¹In ~2.8 day or 67.2 h half-life), zirconium-89 (⁸⁹Zr, ~3.3 day or 78.4 h half-life), iodine-124 (¹²⁴I, 4.17 day half-life), Manganese-52 (⁵²Mn, ~5.59 day half-life), and iodine-131 (¹³¹I, 8.02 day half-life).

In some embodiments, the radioactive isotope is zirconium-89 (⁸⁹Zr), fluorine-18 (¹⁸F), iodine-124 (¹²⁴I), copper-64 (⁶⁴Cu), gallium-68 (⁶⁸Ga), yttrium-90 (⁹⁰Y), or indium (¹¹¹In).

The ectodomain may be directly or indirectly radiolabeled. In some embodiments, the radioactive isotope is connected directly to an amino acid residue of the ectodomain. Direct radiolabeling may be accomplished, for example, through electrophilic substitution at reactive aromatic amino acids (e.g., tyrosine and histidine residues). Direct radiolabeling methods and techniques are known in the art. See, for example, U.S. Pat. Nos. 4,361,544, 4,427,646, 5,985,240, 8,137,540, and 11,298,433, and U.S. Patent Publication Application 2005/0013778.

In some embodiments, the radioactive isotope is connected indirectly to the ectodomain. In some embodiments, the radioactive isotope is connected indirectly to the ectodomain by a chelate. In some embodiments, the chelate is desferrioxamine (DFO) (N-(5-(3-((5-Aminopentyl)hydroxycarbamoyl)propionamido)pentyl)-3-((5-(N-hydroxyacetamido)pentyl)carbamoyl)propionohydroxamic acid), dodecane tetraacetic acid (DOTA) (2,2′,2″,2′″-(1,4,7,10-tetraazacyclododecane-1,4,7,10-tetrayl)tetraacetic acid), 1,4,7-Triazacyclononane-1,4,7-triacetic acid (NOTA), [diethylenetriaminepentaacetic acid (DTPA) (N,N-Bis(2-(bis-(carboxymethyl)amino)ethyl)-glycine), or ethylenediaminetetraacetic acid (EDTA) 2-[2-[bis(carboxymethyl)amino]ethyl-(carboxymethyl)amino]acetic acid, desferrioxamine B (Df) (N1-(5-Aminopentyl)-N1-hydroxy-N4-(5-(N-hydroxy-4-((5-(N-hydroxyacetamido)pentyl)amino)-4-oxobutanamido)pentyl)succinamide), or succinylated-derivatives thereof. The chelate may be bonded to a free —COOH, —NH2, or —SH group on an amino acid residue side chain.

Additional radioactive isotope chelates detectable via PET and SPECT are known in the art. See, for example, U.S. Pat. Nos. 4,479,930, 4,668,503, 4,986,979, 8,309,300, 8,436,147, 8,968,741, 9,000,130, 9,486,537, and 10,517,945, and Vosjan et al., Nat. Protoc. 5(4):739-43 (2010), Calcagno and Fayad, Q. J. Nucl. Med. Mol. Imaging 64(1):74-84 (2020), Boughdad et al., J. Immunother. Cancer. 9(10):e003594 (2021), Boursier et al., Eur. J. Nucl. Med. Mol. Imaging 49(4):1433-1434 (2022), and Toner et al., Sci. Rep. 12(1):6185 (2022).

In some embodiments, the radioactive isotope is connected indirectly to the ectodomain by pre-labeled reagents, in which the reagent is chemically modified with the radioactive isotope (e.g., iodinated) and purified, and then connected to the ectodomain.

In some embodiments, the radioactive isotope is connected to a chelate and the chelate is connected to the radiolabeled tracer by an enzymatic reaction. In some embodiments, the enzymatic reaction is facilitated by a sortase. Sortases are bacterial transpeptidases that recognize an LPXTG (SEQ ID NO: 18) motif where X is any amino acid. Sortase cleaves the peptide bond between the T and G forming an acyl intermediate, which, upon addition of a GGG-R1 substrate, forms the final protein-LPXTGGG-RI product, where R1 can be any biomolecule of interest (See, Mazmanian et al., Science 285(5428):760-763 (1999) and Dorr et al., Proc. Natl. Acad. Sci. U.S.A. 111(37):13343-13348 (2014)). In some embodiments, R1 is DFO, DOTA, NOTA, 3,4,3-(LI-1,2-HOPO), NODAGA, PCTA, transcyclooctene, azide, alkyne, or tetrazine. In some embodiments, the sortase recognition motif is LPETG (SEQ ID NO: 47).

The sortase radiolabeling method is site-specific, stoichiometric, rapid, robust, reproducible, and produces high yields. Non-site-specific approaches for radiolabeling of full-sized antibodies and biomolecules, commonly done via random installation of chelators and radioisotopes via either NHS-lysine or Maleimide-Cysteine chemistry, can be a challenge for small protein tracers, as the random installment of radioisotopes can damage their binding properties. Connection of the radioactive isotope to the ectodomain with sortase ensures minimal perturbation of the ectodomain.

Sortase enzymes and methods of use thereof are known in the art. See, e.g., U.S. Pat. Nos. 8,940,501, 9,751,945, 10,081,684, 10,202,593, 10,471,099, 11,453,870, and 11,542,488, and U.S. Patent Application Publication Nos. 2015/0284477, 2017/0226495, 2019/0256818, and 2021/0238571.

### Dimerization Domains

In some embodiments, the radiolabeled tracer contains a dimerization domain. In these cases, the radiolabeled tracer forms, and is administered in the form of an ectodomain-dimerization domain homodimer. The homodimer thus contains two radiolabeled tracer entities. The order of the ectodomain and the dimerization domain is not critical.

In some embodiments, the dimerization domain may be from IgA, IgD, IgG, IgM, or IgE. In some embodiments, the dimerization domain is the IgG1 constant heavy 3 (CH3) domain. The amino acid sequence of a representative IgG1 CH3 domain is set forth below (SEQ ID NO: 19):

| | |
|---|---|
| 1 | epkspksadk thtapqprep qvytlppsrd eltknqvslt clvkgfypsd iavewesngq |
| 61 | pennykttpp vldsdgsffl yskltvdksr wqqgnvfscs vmhealhnhy tqkslslspg |
| 121 | k |

In some embodiments, the dimerization domain is the IgG1 constant heavy 2 (CH2) domain. The amino acid sequence of a representative IgG1 CH2 domain is set forth below (SEQ ID NO: 20):

| | |
|---|---|
| 1 | pcpapellgg psvflfppkp kdtlmisrtp evtcvvvdvs hedpevkfnw yvdgvevhna |
| 61 | ktkpreeqyn styrvvsvlt vlhqdwlngk eykckvsnka lpapiektis kak |

### Linkers

In some embodiments, the radiolabeled tracer further contains a linker disposed between the ectodomain and the dimerization domain. The linker may provide flexibility in terms of allowing the ectodomain to bind to its respective cognate receptor on the CAR immune cell.

A linker contains at least four amino acids, the selection of which is not critical.

In some embodiments, the linker comprises a tetrapeptide having the sequence GGGX, or a pentapeptide having the amino acid sequence GGGGX (SEQ ID NO: 21), or GSSGSX (SEQ ID NO: 22), where X is any amino acid, or repeating sequences thereof. In some embodiments, X is either cysteine (C) or serine (S). In some embodiments, the linker contains one or more repeats of a tetrapeptide (e.g., repeats of GGGX). In some embodiments, the linker contains one or more repeats of a pentapeptide (e.g., repeats of SEQ ID NO: 20). In some embodiments, the linker has the amino acid sequence GGGGS (SEQ ID NO: 23), GSPRG (SEQ ID NO: 24), GGGGSGGGGS (SEQ ID NO: 25), GGGGSGGGGSGGGGS (SEQ ID NO: 26), GGGGSGGGGSGGGGSGGGGS (SEQ ID NO: 27), GSPRGGGGSGGGGSGGGGS (SEQ ID NO: 28), GSTSGSGKPGSGEGSTKG (SEQ ID NO: 29), KESGSVSSEQLAQFRSLD (SEQ ID NO: 30), EGKSSGSGSESKST (SEQ ID NO: 31), or GSAGSAAGSGEF (SEQ ID NO: 32).

In some embodiments, the linker may be from IgA, IgD, IgE, IgG, or IgM. In some embodiments, the linker may be from the hinge region of CD3ζ, CD4, CD8α, CD28, IgG1, IgG2, or IgG4. Amino acid sequences of representative linkers are listed in Table 2.

TABLE 2
Amino Acid Sequences of Representative Linkers

| Linker | Sequence |
|---|---|
| CD3ζ | QSFGLLDPK |
| (SEQ ID NO: 33) | |
| CD4 | LSEGDKVKMDSRIQVLSRGVNQT |
| (SEQ ID NO: 34) | |
| CD8α | KPTTTPAPRPPTPAPTIASQPLS |
| (SEQ ID NO: 35) | KRPEACRPAAGGAVHTRGLDFAC |
| | DIY |
| CD28 | IEVMYPPPYLDNERSNGTIIHVK |
| (SEQ ID NO: 36) | GKHLCPSPLFPGPSKP |
| IgG1 | EPKSCDKTHTCPPCPAPELLGG |
| (SEQ ID NO: 37) | |
| IgG2 | ERKCCVECPPCPAPPAAA |
| (SEQ ID NO: 38) | |
| IgG4 | ESKYGPPCPPCPAPEFLGG |
| (SEQ ID NO: 39) | |

In some embodiments, the radiolabeled tracer contains a purification tag. A purification tag typically entails addition of in-frame nucleic acids that may be translated into amino acids along with the protein to which the tag is attached. The purification tag allows the protein to be captured or detected. Representative examples of purification tags include polyhistidine, FLAG epitope, histidine affinity tag (HAT), herpes simplex virus (HSV) epitope, human influenza hemagglutinin (HA), glutathione S-transferase (GST), KT3 epitope, maltose binding protein (MBP), AU1 epitope, AU5 epitope, Bacteriophage T7 epitope, and myc tags. Amino acid sequences of representative purification tags are listed in Table 3.

TABLE 3
Amino acid Sequences of
Representative Purification Tags

| Purification Tag | Sequence |
|---|---|
| AU1 epitope | DTYRYI |
| (SEQ ID NO: 40) | |
| AU5 epitope | TDFYLK |
| (SEQ ID NO: 41) | |
| Polyhistidine | HHHHHHH |
| (SEQ ID NO: 42) | |
| FLAG epitope | DYKDDDDK |
| (SEQ ID NO: 43) | |
| FLAG His tag | EFDYKDDDDKGSLPETGGHHHHHH |
| (SEQ ID NO: 44) | |
| HAT | KDHLIHNVHKEFHAHAHNK |
| (SEQ ID NO: 45) | |
| HSV epitope | QPELAPED |
| (SEQ ID NO: 46) | |

In some embodiments, the radiolabeled tracer is further conjugated to a polyethylene glycol (PEG) polymer, chain, or molecule (i.e., it is PEGylated). PEG is a synthetic, hydrophilic, non-toxic, FDA-approved, non-immunogenic, polymer with widespread use in biomedical and other applications and may decrease non-specific kidney uptake and enhance image signal-to-noise ratios. See, e.g., Rashidian et al., J. Exp. Med. 214(8):2243-2255 (2017) and Teunissen et al., Star Protoc. 2(2):100434 (2021).

In some embodiments, the conjugated PEG molecule has a molecular weight of about 5 kilodaltons (kDa), about 10 kDa, about 15 kDa, or about 20 kDa. The PEG molecule may be conjugated to any moiety of the radiolabeled tracer (i.e., at least one of the ectodomain, linker, and dimerization domain) using standard laboratory techniques such as, for example, azide-alkyne click chemistry.

### Diagnostic Compositions

In one aspect, the present disclosure provides a diagnostic composition which includes an effective amount of the radiolabeled tracer and a pharmaceutically acceptable carrier.

The term “effective amount of the radiolabeled tracer” as used herein refers to a sufficient amount of the radiolabeled tracer to provide the desired effect.

Diagnostic compositions may be provided as sterile lyophilized solid or liquid preparations. Lyophilized solid compositions may be prepared by freezing a liquid composition and removal of the frozen liquid by sublimation. Lyophilized solid preparations may be reconstituted and diluted into a liquid preparation before use. Pharmaceutically acceptable liquid carriers include aqueous or non-aqueous carriers alike. Representative examples of liquid carriers include sterile water for injection, saline, Lactated Ringer Injection solution, buffered saline, phosphate buffered saline (PBS), a soluble protein, soluble sugars (e.g., dextrose), dimethyl sulfoxide (DMSO), polyol (e.g., glycerol, propylene glycol, liquid polyethylene glycol, and the like), ethanol, and suitable mixtures thereof. In some embodiments, the liquid carrier includes a protein dissolved or dispersed therein, representative examples include serum albumin (e.g., human serum albumin, recombinant human albumin), gelatin, and casein. The compositions are typically isotonic, i.e., they have the same osmotic pressure as blood. Citric acid, sodium chloride, sugars, polyalcohols, and isotonic electrolyte solutions (e.g., Plasma-Lyte®) may be used to achieve the desired isotonicity. Depending on the carrier and the immunoctyokine, other excipients may be added, e.g., wetting, dispersing, or emulsifying agents, gelling and viscosity enhancing agents, preservatives and the like as known in the art. In some embodiments, the compositions include citric acid, ethylenediaminetetraacetic acid (EDTA), and polysorbate 20 with a pH range between about 6.8 to about 7.2.

### Kits

Diagnostic compositions may be assembled into kits or diagnostic systems for use in imaging CAR immune cells or in treating cancer after CAR immune cell therapy. The kits or diagnostic systems may include one or more dosage formulations containing an effective amount of the radiolabeled tracer disposed in a suitable container, e.g., tube, vial, ampoule, bottle, syringe, or bag, and printed instructions for use in administering the composition to a subject having cancer. In some embodiments, the subject received CAR immune cell therapy, and wherein detection of the radioactive isotope in the subject may be indicative of persistence of CAR immune cell therapy. In some embodiments, the tracer is present in the suitable container formulated in a pharmaceutically acceptable liquid carrier. The kits or diagnostic systems may also include one or more dosage formulations of an appropriate therapy for cancer and/or stimulating CAR immune cells. The appropriate therapy for cancer and/or stimulating CAR immune cells may be disposed in a pharmaceutically acceptable carrier further disposed in a suitable carrier. The kits or diagnostic systems of the disclosure may also comprise printed instructions for using the additional components.

### Methods of Use

In some aspects, the present disclosure is directed to detecting CAR immune cells in a subject. The method entails administering to a subject in need thereof, a diagnostic composition containing an effective amount of the radiolabeled tracer, where prior to the administering of the diagnostic composition, the subject has been administered an effective number of CAR immune cells, and producing a first image of a body area of the subject by PET or SPECT imaging, wherein detecting the radioactive isotope in the body tissue is an indication of the CAR immune cells in the body area.

The term “subject” (or “patient”) as used herein includes all members of the animal kingdom suffering from cancer or a disease treatable by CAR immune cells. In some embodiments, the subject is a human. Therefore, a subject “having CAR immune cell administration” or “in need of” imaging according to the present disclosure broadly embraces subjects who have been positively diagnosed with cancer, including subjects having active cancer who have been previously treated with one or more rounds of CAR immune cell therapy.

The effective amount of a radiolabeled tracer for a given patient varies depending one or more factors that may include the age, body weight, type, and general health of the subject. Ultimately, the attending physician will decide the appropriate dose and dosage regimen. Typically, the radiolabeled tracer may be given in a single dose before imaging of the subject. In some embodiments, the effective amount of the radiolabeled tracer is about 37 megabecquerel (MBq) (about 1 mCi) to about 148 MBq (about 4 mCi) per dose. In some embodiments, the effective amount of the radiolabeled tracer is about 0.5, about 1, about 2, about 4, about 5, about 6, about 8, about 10, or about 20 mg protein per dose. In some embodiments, the effective amount of the radiolabeled tracer is about 2 mg to about 8 mg protein per kg of subject weight per dose.

The term “effective number of CAR immune cells” (which indirectly includes a corresponding amount of a CAR) as used herein refers to a sufficient number of the CAR immune cells to provide the desired effect.

The number of CAR immune cells administered to a subject may vary between wide limits, depending upon factors such as the location, type, and severity of the cancer, the age, body weight, and condition of the individual to be treated. In some embodiments, the effective number of the CAR immune cells is between approximately 1×10⁵ to approximately 1×10¹⁰ cells per subject. In some embodiments, the effective number of the CAR immune cells is between approximately 1×10⁴ to approximately 6×10⁸ cells per kg of subject body weight. Typically, the CAR immune cells will be given in a single dose.

Compositions containing an effective amount of the radiolabeled tracer and an effective number of the CAR immune cells may be administered to a subject for the treatment of a cancer by any medically acceptable route. In some embodiments, the radiolabeled tracers and CAR immune cells are delivered intravenously, although they may also be introduced into other convenient sites (e.g., to an affected organ or tissue) or modes, as determined by an attending physician. In some embodiments, the radiolabeled tracer is administered intratumorally.

Expansion and differentiation agents can be provided prior to, during or after administration of the cells to increase differentiation, expansion, and/or persistence of the CAR immune cells (e.g., T cells and NK cells).

Administration of the CAR immune cells may be autologous or allogeneic. For example, immune cells or progenitors thereof can be isolated from a tissue of body fluid from one subject prior to administration to the same subject (autologous) or a different, compatible subject (allogeneic).

### Administration

The methods of the present disclosure entail the administration of a diagnostic composition to a cancer patient who had received a prior administration of CAR immune cells. The radiolabeled tracer is administered to the subject to determine if the CAR immune cells have lost vitality or persistence in the subject, referred herein as a suboptimal response.

In some embodiments, administration of the radiolabeled tracer is conducted at least about 1 week, at least about 2 weeks, at least about 1 month, at least about 2 months, at least about 3 months, at least about 6 months, at least about 9 months, at least about a year, or more than a year after administering the CAR immune cells.

In some embodiments, the radiolabeled tracer is administered once every 3 weeks (a 21-day cycle) as an infusion over about 30 to about 90 minutes. In some embodiments, the radiolabeled tracer is administered for 5 consecutive days every 21 days and repeated for 8 cycles.

### Imaging and Image Analysis

In some embodiments, the imaging is conducted via PET. PET scanners and techniques are known in the art. See, for example, U.S. Pat. Nos. 8,660,636, 9,495,771, 10,909,731, 11,069,098, and 11,300,695.

In some embodiments, the PET imaging is conducted via PET-CT. PET-CT scanners and techniques are known in the art. See, for example, U.S. Pat. Nos. 7,507,968, 7,569,829, 7,813,535, 7,869,860, 8,063,376, and 9,155,514.

In some embodiments, the PET imaging includes concomitant use of MRI (PET-MRI). Combined PET-MRI techniques are known in the art. See, for example, U.S. Pat. Nos. 4,939,464, 7,835,782, 7,888,632, 8,073,525, 8,073,527, 8,452,378, 8,467,845, 9,459,333, 9,510,797, 9,555,135, and 10,627,466 and Senders et al., J. Am. Coll. Cardiol. 71(3):321-335 (2018).

In some embodiments, the imaging is conducted via SPECT. SPECT scanners and techniques are known in the art. See, for example, U.S. Pat. Nos. 7,109,489, 7,635,848, 7,683,332, 7,968,851, 8,421,021, and 8,462,911.

Image analysis techniques for these imaging modalities are known in the art. See, for example, U.S. Pat. Nos. 7,251,523, 7,876,938, 9,204,835, 9,846,922, 10,482,600, 10,943,681, 11,055,847, 11,164,045, 11,222,243, 11,301,995, and 11,424,035.

The present radiolabeled tracer compositions and methods may detect CAR immune cells in any bodily organ or tissue and may involve any organ in which the cancer is believed to be present or the CAR immune cells are believed to be present. In some embodiments, the imaged body area is bone marrow, spleen, lymph node tissue, lung, liver, central nervous system (CNS) (also referred as brain or head), peritoneum, chest (i.e., torso), abdominal segment (i.e., mid-section or midriff), head, tumor tissue (including primary tumors, and any metastatic lesions), a combination thereof, the entire body, or close to the entire body (e.g., entire body without imaging the head or other extremities).

The term “tissue” as used herein refers to a group of cells and intercellular matrix that have similar structure and that function together as a unit. An image of a tissue embraces taking an image of the entire organ in which the tissue resides. For example, an image of splenic tissue embraces taking an image of the spleen.

In some embodiments, the imaged body area is bone marrow. In some embodiments, the imaged body area is tumor tissue. In some embodiments, the imaged body area includes bone marrow and tumor tissue. In some embodiments, the imaged body area is close to the entire body.

The image of the subject's body area is taken after administration of the radiolabeled tracer. In some embodiments, the image is taken about 2 hours to about 96 hours after administering the radiolabeled tracer. In some embodiments, the image is taken about 2 hours to about 24 hours after administering the radiolabeled tracer. In some embodiments, the image is taken about 2 hours to about 5 hours after administering the radiolabeled tracer. In some embodiments, the image is taken on the same calendar day as the radiolabeled tracer is administered.

In some embodiments, the method of imaging CAR immune cells in a subject entails identifying a first location of elevated radiolabeled tracer within the image relative to a control image of a body area from a subject that has not received CAR immune cells. The first location of elevated radiolabeled tracer may be manually identified, commonly referred to as a region of interest (ROI) analysis or by automated methods, commonly referred to as a voxel-based analysis. See, Snook et al., Neuroimage 34(1):243-52 (2007).

The ROI analysis method may involve manually defining an area of elevated tracer in the image within which to make measurements (i.e., quantify the amount of radioactive isotope within the image or location). The voxel-based analysis method involves a voxel-by-voxel statistical comparisons throughout the image. The voxel-based analysis method spatially normalizes each set of images to a control image, and thus assigning an “address” to each voxel. Then, a voxel-by-voxel statistical comparison of the images can be performed. This voxel-by-voxel statistical comparison can be used to quantify the amount of radioactive isotope within the image or location.

Radiolabeled tracers used in PET and SPECT emit γ radiation, which is detected by a PET or SPECT detector, as known in the art. The resulting data on the emitted radiation collected by the detector is converted into voxel data to form an image. Methods for converting detector data into voxel data are known in the art, for example, by the filtered back projection method. A voxel represents a value on a regular grid in three-dimensional space. Each voxel in the image records the intensity of γ radiation received by the detector.

The image may be evaluated, for example, by a nuclear radiologist, to determine if the radiolabeled tracer is present in the imaged body area. A level of radioisotope greater than the background present in one or more control images may be indicative of inflammation in the imaged tissue. See, for example, Calcagno and Fayad, Q. J. Nucl. Med. Mol. Imaging 64(1):74-84 (2020).

A measured signal in one or both of the tumor tissue and bone marrow may indicate that the CAR immune cells are responding to the tumor and may predict that the subject will respond to the CAR immune cell therapy.

In some embodiments, the subject is suspected of having a toxicity related to CAR immune cell therapy, referred herein as “CAR immune cell toxicity.” representative examples of such toxicities include cytokine release syndrome, immune cell-associated neurotoxicity syndrome (ICANS), acute anaphylaxis, tumor lysis syndrome (TLS), on-target off-tumor recognition, and anaphylaxis. In some embodiments where the subject is suspected of having a CAR immune cell toxicity, the imaged body area is the CNS, chest, and/or the liver.

CRS and ICANS clinical toxicity may be graded by standard techniques. For example, CRS grading may be performed according to the Lee criteria or the American Society for Transplantation and Cellular Therapy (ASTCT) consensus criteria (Lee et al., Biol. Blood Marrow Transplant. 25(4):625-638 (2019), Atkinson et al., Support. Care Cancer. 24(8):3669-3676 (2016), US Department of Health and Human Services. “National Institutes of Health NCI: Common Terminology Criteria for Adverse Events (CTCAE) v5.0 (2017)). ICANS may be graded according to the CAR T-Cell Therapy-Associated Toxicity scale (Lee et al., Biol. Blood Marrow Transplant. 25(4):625-638 (2019), Lee et al., Blood 126(8):188-195 (2015)).

The detected radioactive isotope in the imaged body area is an indication of the CAR immune cells presence or number in the body area. The signal emitted from the radioactive tracer bound to the CAR immune cells may serve as a specific indication of the presence or amount CAR immune cells relative to the number of CAR immune cells that were administered to the subject. While not being strictly quantitative in terms of determining the exact number of CAR immune cells, the strength (or weakness) of the radioactive (e.g., PET) signal may be correlated to number and persistence of the CAR immune cells and therapeutic outcome.

For example, a relatively high signal may indicate with a better or more robust persistence of the CAR immune cells which may reflect the presence of a high number of CAR immune cells relative to the number of cells that had been administered to the patient. This result may be interpreted (e.g., by an attending physician, e.g., clinical oncologist) as prognostic of a better therapeutic outcome.

Conversely, a relatively low signal may indicate a lesser or non-robust persistence of the CAR immune cells which may reflect the presence of a low number of CAR immune cells relative to the number of cells that had been administered to the patient.

Generally, a very weak or lack of a substantive signal of the radioactive tracer in the imaged body area (e.g., the lymphoid organs of the spleen, lymph nodes, bone marrow, or a combination thereof) may indicate that the subject has had or is having a suboptimal response. In such cases, an attending physician may advise additional tests to assay the CAR immune cells, or additional therapies to stimulate the CAR immune cells. A lack of signal in the imaged body area may indicate that the CAR immune cells did not persist in the patient, and that the patient is at a high chance of relapse. In this case, the attending physician may advise additional anti-cancer therapies, including for example, an additional administration of the CAR immune cells.

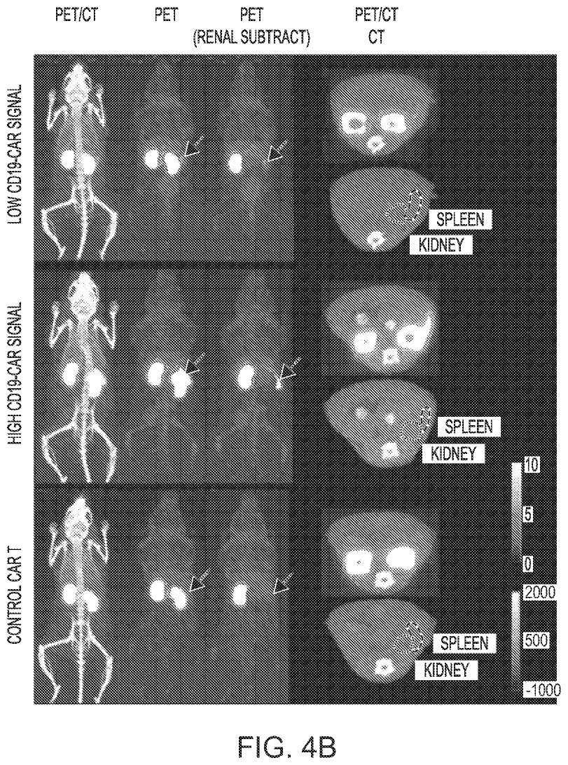

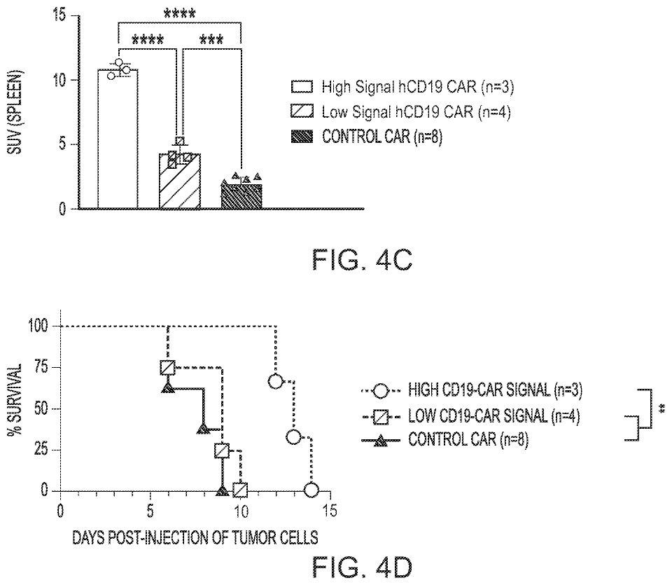

A suboptimal response may be determined by known methods. For example, the presence or amount of signal can be compared over time in one subject or compared to standardized signal in a patient population. Standardized signal in patient populations may then be correlated with optimal or suboptimal response to a given therapy. A representative correlation of standardized uptake value (SUV) with optimal and suboptimal responses is shown in the working examples disclosed herein. FIG. 4A-FIG. 4D illustrate data showing that patients with signal SUVs below a threshold of 10 or less had a suboptimal response to CAR immune cell therapy whereas patients with signal SUVs above the threshold of 10 had an optimal response to CAR immune cell therapy.

### Longitudinal Studies

Repeated imaging of the same subject over a period of time, referred to as a longitudinal study, can be used to monitor the progression of a disease or evaluate the effectiveness of a treatment. The methods disclosed herein broadly embrace longitudinal studies involving one or more subsequent administrations of the radiolabeled tracer and imaging of the subject (for a total of two or more administration and imaging events).

In some embodiments, the method further entails administering a second effective amount of the radiolabeled tracer after the first image is produced, producing a second image of the body area. The time period between the first and second administration of the radiolabeled tracer may be determined by an attending physician. In some embodiments, the second image is produced at least two weeks after the first image was produced. In some embodiments, the second image is produced at least one month, at least two months, at least three months, at least six months, at least a year, or more than a year after the first image was produced.

### Cancer

In some aspects, the present disclosure is directed to treating a cancer in a subject. The method entails determining the presence of CAR immune cells in a body area of a subject by administering to the subject the diagnostic composition containing the radiolabeled tracer, where prior to the administering of the diagnostic composition, the subject had been administered an effective number of CAR immune cells, producing a first image of a body area of the subject by PET or SPECT imaging, wherein detecting the radioactive isotope in the body area is an indication of the CAR immune cells in the body area, and administering an appropriate therapy to the subject following a determination that the presence or number of CAR immune cells detected is suboptimal (e.g., as disclosed above).

The terms “treat”, “treating”, and “treatment” as used herein refer to any type of intervention, process performed on, or the administration of an active agent to the subject in need thereof (e.g., a subject who had received, is receiving, or will receive CAR immune cells) with the therapeutic objective (“therapeutic effect”) of reversing, alleviating, ameliorating, inhibiting, diminishing, slowing down, arresting, stabilizing, or preventing the onset, progression, development, severity or recurrence of a symptom, complication or condition, or biochemical indicia associated with a cancer.

Cancers that may be treated in accordance with the disclosed method include both hematopoietic cancers and cancers characterized by the presence of a solid tumor. In some embodiments, the cancer is a hematopoietic cancer. Representative hematological cancers include plasma cell neoplasm (e.g., myeloma, multiple myeloma, relapsed or refractory multiple myeloma, plasma cell myeloma, extramedullary multiple myeloma, monoclonal gammopathy of unknown significance (MUGS), asymptomatic smoldering multiple myeloma, or solitary plasmacytoma), lymphoma (e.g., Hodgkin's lymphoma, non-Hodgkin's lymphoma, Burkitt's lymphoma, plasmablastic lymphoma, plasmacytoid lymphoma, or diffuse large B-cell lymphoma), leukemia (e.g., relapsed or refractory B cell acute lymphocytic leukemia (B-ALL), or relapsed or refractory acute lymphoblastic leukemia), and carcinomas (e.g., Waldenstrom macroglobulinemia or glioblastoma (astrocytoma)). In these embodiments, the therapeutic effect might include on or more art-recognized indicia of therapeutic efficacy, representative examples of which include prevention or prolongation of metastases, improvement in survival time, total/complete or partial remission of a cancer, e.g., no detectable cancer cells and less tumor cells or smaller tumors, respectively, or a reduction in tumor cell number. In some embodiments, the hematopoietic cancer is multiple myeloma, lymphoma, or leukemia.

In some embodiments, the cancer is characterized by the presence of a solid tumor. In some embodiments, the cancer is a bladder cancer (e.g., transitional cell carcinoma, also called urothelial carcinoma), pancreatic cancer (e.g., pancreatic ductal adenocarcinoma (PDAC)), kidney cancer (e.g., renal cell carcinoma (RCC), kidney renal clear cell carcinoma (KIRC), transitional cell cancer, or Wilms tumor), skin cancer (e.g., melanoma, skin cutaneous melanoma (SKCM), basal cell carcinoma, and squamous cell carcinoma of the skin), lung cancer (e.g., small cell lung cancer, non-small cell lung cancer, including lung adenocarcinoma (LUAD) and lung squamous cell carcinoma (LUSC)), head and neck cancer (e.g., squamous cell carcinoma of the head and neck (SCCHN) also called head and neck squamous cell carcinoma (HNSC), laryngeal and hypopharyngeal cancer, nasal cavity and paranasal sinus cancer, nasopharyngeal cancer, oral and oropharyngeal cancer, and salivary gland cancer), colon or rectal cancer (e.g., colorectal carcinoma (CRC), colon adenocarcinoma (COAD), rectum adenocarcinoma (READ)), ovarian cancer (e.g., cystadenocarcinoma, ovarian embryonal carcinoma, ovarian adenocarcinoma, epithelial ovarian carcinomas, fallopian tube cancer, and primary peritoneal cancer), endometrial cancer, cervical cancer (e.g., cervical squamous cell carcinoma and endocervical adenocarcinoma (CESC)), prostate cancer (e.g., prostate adenocarcinoma (PRAD)), and stomach cancer (e.g., stomach adenocarcinoma (STAD)).

### Appropriate Cancer Therapy

The methods of the present disclosure entail administration of an appropriate therapy to a cancer patient who had received a prior administration of immune cells containing a CAR that contains an extracellular domain that binds the ectodomain of the radiolabeled tracer. The term “appropriate therapy” as used herein refers to any therapy which beneficially treats the cancer or CAR immune cell toxicity.

The appropriate therapy is administered to the subject subsequent to a determination that the subject has had a suboptimal response to the CAR immune cell administration.

The appropriate therapy may be administered once the imaged immune cell presence is less than the administered immune cell presence. In some embodiments, the appropriate therapy is administered once the measured immune cell presence is quantified and is less than 90%, less than 80%, less than 70%, less than 60%, less than 50%, less than 25%, less than 10%, or less than 5% of the administered presence of immune cells.

The appropriate therapy may be administered once the imaged immune cells are located in a specific body area. In some embodiments, the appropriate therapy is administered once the immune cells are not detected in the tumor tissue. In some embodiments, the appropriate therapy is administered once the immune cells are detected in the bone marrow. In some embodiments, the appropriate therapy is administered once the immune cells are detected in the bone marrow and detected not the tumor tissue.

Additional therapies to stimulate the CAR immune cells may include, for example, immunotherapy, immune cell regulatory protein inhibition, TGF-β inhibition, or a combination thereof.

Immunotherapy utilizing immune checkpoint inhibitors may be used to overcome immune cell exhaustion and stimulate the CAR immune cells. Immune checkpoint molecules include, for example, PD-1, PDL1, CTLA4, KIR, TIGIT, TIM-3, LAG-3, BTLA, VISTA, CD47, and NKG2A. See, Adusumilli et al., Cancer Discov. 11(11):2748-2763 (2021) and NIH clinical trials NCT03310619, NCT03630159, and NCT02926833. Clinically available examples of immune checkpoint inhibitors include durvalumab (Imfinzi®), atezolizumab (Tecentriq®), and avelumab (Bavencio®). Clinically available examples of PD-1 inhibitors include nivolumab (Opdivo®), pembrolizumab (Keytruda®), and cemiplimab (Libtayo®). Additional inhibitors that may be useful in the practice of the present disclosure are known in the art. See, e.g., U.S. Patent Application Publication Nos. 2012/0321637, 2014/0194442, and 2020/0155520.

Additional Immunotherapy agents that may also be used to stimulate CAR immune cells include cereblon-modulating agents (e.g., avadomide and iberdomide), BTK inhibitors (e.g., ibrutinib and acalabrutinib; see NIH clinical trials NCT03960840, NCT01865617, NCT02640209, and NCT03331198), hypomethylating agents (see NIH clinical trials NCT02851589, NCT04109482, NCT03017131), PI3K inhibitors (see NIH clinical trial NCT03274219), y-secretase inhibitors (see NIH clinical trial NCT03502577), lenalidomide (see NIH clinical trial NCT03070327), and rituximab.

Inhibitors of immune cell regulatory proteins that may be used to stimulate CAR immune cells include, for example, RNA interference (RNAi) inhibitors (e.g., miRNA, siRNA, shRNA, or antisense oligonucleotides) targeting thymocyte selection associated high mobility group box (TOX) 1, TOX4, nuclear receptor subfamily 4 group A member 1 (NRA1), NRA2, and NRA3. See, Khan et al., Nature 571(7764):211-218 (2019); Kim et al., Genome Med. 12(1):22 (2020); Seo et al., Proc. Natl. Acad. Sci. U.S.A 116(25):12410-12415 (2019); Chen et al., Nature 567(7749):530-534 (2019); Liu et al., Nature 567(7749):525-529 (2019). An additional immune cell regulatory protein is casitas B lymphoma-b (Cbl-b) protein. Cbl-b inhibitors include, for example, NTX-801, NX-1607, NX-0255, APN-401, APN-411, HST-1011, and STX-500.

TGF-β inhibitors that may be used to stimulate CAR immune cells include the fusion protein bintrafusp alfa, the antibodies fresolimumab (GC1008), 6.3G9, 264RAD, lerdelimumab, SAR439459, Tβm1, and LY3022859, the small molecule inhibitors galunisertib (LY2157299), LY3200882, and vactosertib, and RNAi inhibitors trabedersen, and belagenpumatucel-L (Lucanix™). See, Jaschinski et al., Curr. Pharm. Biotechnol. 12(12):2203-2213 (2011); Eberlein et al., Oncogene 32(37):4406-4416 (2013); Huynh et al., Biomolecules 9(11):743 (2019); Ciardiello et al., Ann. Oncol. 31(10):1336-1349 (2020).

These and other aspects of the present application may be further appreciated upon consideration of the following Examples, which are intended to illustrate certain embodiments of the application but are not intended to limit its scope, as defined by the claims.

### EXAMPLES

### Example 1: Materials and Methods

Alexa-labeled and radiolabeled tracers (probes) were prepared and tested by standard cloning, protein expression, and purification techniques. Radiolabeled tracers were purified via size-exclusion chromatography and characterized via SDS-PAGE and LC-MS. Three sortase substrates (Gly-Gly-Gly-AlexaFlour647, Gly-Gly-Gly-NOTA and Gly-Gly-Gly-deferoxamine (DFO) were conjugated onto radiolabeled tracers and conjugation was confirmed by SDS-PAGE and LC-MS analyses.

Radiochemistry. For ⁶⁴Cu labeling, a 1.5-mL centrifuge tube was loaded with the NOTA-labeled tracer (about 30 μM tracer in 200 mM NH40Ac buffer (pH 6.5) in a volume of about 300 μL) and ⁶⁴CuCl₂ (about 1.0 mCi) in 200 mM NH40Ac buffer (75 μL, pH 6.5). The tube was sealed and shaken at 37° C. for 30-45 min. The mixture was then analyzed by radio-TLC (ITLC, 50 mM EDTA, pH 7, Rf ⁶⁴Cu/EDTA=1.0, Rf ⁶⁴Cu-tracer=0.0) showing % yield conversion. The mixture was loaded onto a PD-10 size-exclusion cartridge and eluted with PBS to provide ⁶⁴Cu-labeled tracer. For ⁸⁹Zr radiolabeling, a similar approach was followed with some minor changes. Tracers were loaded into centrifuge tubes in 0.5 M HEPES buffer, pH 7.5 and the pH of ⁸⁹Zr⁴⁺ ion stock solution corresponding to ~1.0 mCi is adjusted to ~7 using 2M Na₂CO₃ (Rashidian et al., J. Exp. Med. 214(8):2243-2255 (2017)). Decay-corrected radiochemical yield was calculated following standard calculations.

Cell culture and cell lines. The cell lines utilized include murine B-cell acute lymphoblastic leukemia (B-ALL) cells, murine pancreatic ductal adenocarcinoma (PDAC) cells, and human embryonic kidney 293T cells (HEK293T). HEK293T cells, acquired from the American Type Culture Collection (ATCC), were sustained in DMEM with L-glutamine and sodium pyruvate (Corning, 10-013-CM), supplemented with 15% FBS. Murine B-ALL cells were cultured in RPMI with L-glutamine (Corning, 10-040-CM), supplemented with 10% fetal bovine serum (FBS) and 2-mercaptoethanol to a final concentration of 0.05 mM (Gibco, 21985023). Murine PDAC cells were grown in DMEM with L-glutamine and sodium pyruvate (Corning, 10-013-CM), supplemented with 10% FBS. Routine mycoplasma contamination checks were conducted for all cell lines. Primary murine T cells were procured from mouse spleens and cultured on plates coated with activating antibodies. The T-cell medium (TCM) included of RPMI with L-glutamine (Corning, 10-040-CM), supplemented with 10% FBS, recombinant human IL-2 (rhIL-2, final concentration of 20 ng/mL; Peprotech, Cat #200-02-1 mg), and 2-mercaptoethanol to a final concentration of 0.05 mM (Gibco, 21985023).

Viral supernatant production. Viral supernatant was produced using standard methods. Briefly, HEK293T cells were transfected with retroviral or lentiviral transfer plasmid and packaging vector (retrovirus: pCL-Eco, Addgene, 12371; lentivirus: psPAX2, Addgene, 12260 with vesicular stomatitis virus G protein (VSVg) envelop plasmid pMD2.G, Addgene, 12259; pMMLV hCD19(-ICD), cloned from VectorBuilder plasmid #VB181207-1130jpc) using Mirus TransIT®-LT1 (Mirus, MIR2305) as indicated by the manufacturer. Viral supernatant was collected 48 hours and 72 hours after transfection, passed through a 0.45 μm filter, and stored at 4° C. for a maximum of one day.

The pMMLV hCD19(-ICD) plasmid was produced by deleting most of the ectodomain of hCD19 by PCR from the pMMLLV hCD19 plasmid (VectorBuilder). PCR was performed using Q5 polymerase (NEB M0491S) with forward primer 5′-CCACCACCCAGCTTTCTTGTACAAAGTGGT-3′ (SEQ ID NO: 49) and reverse primer 5′-TCAGGCGGCCGCGACCAGGGCTCTTTGAAGATGAAGA-3′ (SEQ ID NO: 50). The finished product was PCR purified using the NEB Monarch® PCR clean-up kit (NEB T1030S) and ligated using the DNA ligation kit, Mighty Mix (Takara 6023). The validity of the final product was confirmed using Sanger sequencing.

The sequence of the CD19 ectodomain incorporates mutations compared to the wild-type CD19 protein, enhancing its stability (Klesmith et al., Biochemistry 58(48):4869-4881 (2019)). We incorporated a FLAG tag (DYKDDDDK), a sortase recognition motif (LPETG), and a His6 tag at the C-terminus of the protein to generate CD19 ectodomain with amino acid sequence of SEQ ID NO: 48.

Tracer production. All genes underwent codon optimization for HEK293T mammalian expression, followed by synthesis and insertion into a vector expression system equipped with a signal sequence. A stable HEK293T cell line was established through transfection with pPAX2, pVSVG (packaging vectors), and the lentivirus plasmid containing the sequence of interest. Virus harvesting occurred at 48, 72, and 96 hours, followed by sedimentation at 20,000×g for 2 hours and resuspension in optiMEM™ media. Fresh HIEK293T cells were then transduced with the virus and allowed to recover in DMEM complete media. Puromycin selection was applied to retain cells that integrated the lentivirus plasmid. After expansion, cells transitioned to serum-free media for 48-72 hours before commencing supernatant harvesting.

Post-collection, protein expression in the supernatant was validated via SDS-PAGE. Subsequently, proteins underwent purification using a nickel nitrilotriacetic acid (Ni-NTA) metal affinity column. Non-specifically bound proteins were eliminated through washing with a low-concentration imidazole solution (40 mM), and the protein of interest was recovered using a high-concentration imidazole solution (400 mM). The final purification step involved size-exclusion chromatography, and the purified proteins were stored in 50 mM HEPES buffer, pH 7.5, at −80° C. until use.

The DFO-labeled CD19 with PEG 20 kDa was prepared following our established procedures (Rashidian et al., J. Exp. Med. 214(8):2243-2255 (2017)). For DFO labeling, 1 μL of 50 mM HEPES with 150 mM NaCl (pH 7.5) underwent Chelex® treatment for 16 hours at 4° C. using 10 g Chelex® 100 sodium form beads to eliminate trace metals. Next, the protein and all reagents, including sortase, were dialyzed in trace metal-free HEPES using Thermo Scientific SnakeSkin™ Dialysis Tubing for an additional 16 hours. Post-dialysis, GGG-azide-DFO substrate, dissolved in DMSO was added to the protein at a concentration of 2 mM. Subsequently, Sortase 7M was included, reaching a final concentration of 7.5 μM in a 1.5 ml Eppendorf tube. The sortase-mediated labeling reaction proceeded overnight at 4° C. Removal of excess sortase and unreacted protein was achieved using Ni-NTA beads. To eliminate excess DFO, a PD-10 column was employed. Finally, 20 kDa PEG DBCO was introduced to the protein solution for installation on the radiolabeled tracer through a click reaction. The success of PEGylation onto the protein was confirmed via SDS-PAGE analysis.

CAR T cell production. Briefly, CD8+ T-cells were isolated from the spleens of 14-week-old male or female C57BL/6 mice (Jackson Laboratory) using Miltenyi Biotec CD8a (Ly-2) MicroBeads for mouse (positive selection kit; Miltenyi, 130-117-044) and LS columns (Miltenyi, 130-042-401) as per the manufacturer's instructions. The isolated T cells were cultured at 1×10⁶ cells/mL on 6-well plates coated with anti-murine CD3F and anti-murine CD28 activating antibodies (Bio X-Cell, BE0001-1 and BE0015-1) in T cell media.

After 24 hours, activated T cells were collected, counted, and resuspended at 0.5×10⁶ in a 50:50 mixture of fresh T cell media with viral supernatant supplemented with protamine sulfate to a final concentration of 10 g/mL (MS Biomedicals, ICN19472910). The cells were spin-infected at 1000×g for 1.5 hours at 37° C. on new antibody-coated plates. The next day, T cells were again collected, counted, and resuspended at 1×10⁶ cells/mL in fresh T cell media, re-plated on new antibody-coated plates. Lastly, 24 hours later, T cells were collected, counted, and the percentage of CAR⁺, GFP⁺ T cells determined by flow cytometry. The desired number of CAR T cells was then prepared for injection by resuspension in saline and injected via tail vein. Alternatively, T cells were resuspended at this step in T cell media and plated for in vitro killing assays.

In vitro killing assays. Briefly, target cells were counted and co-cultured with or without CAR T cells at specified Effector-to-Target (E:T) ratios, accounting for the CAR-T transduction efficiency, in TCM. After approximately 24 hours, the cell suspension was subjected to flow cytometry analysis to evaluate live/dead status (via DAPI stain), % hCD19⁺ cells (anti-human CD19 BV785, BioLegend), and % CD8+ cells (anti-mouse CD8-PE/Cy7; BioLegend #100722). Flow cytometry was also employed to determine the densities of each cell type (CAR-T, target cell, non-transduced T-cell). The resulting target cell densities in CAR-T-containing wells were normalized to those in control wells, seeded with the same number of target cells but with control CAR-T or non-transduced T cells. All flow cytometry experiments were conducted with a minimum of 10,000 live cells (via DAPI exclusion) and subsequent data analysis.

Interferon-γ (IFN-γ) release ELISA assays. The enzyme-linked immunosorbent assay (ELISA) followed standard procedures. Briefly, supernatants from in vitro CAR-T killing assays were collected and centrifuged to eliminate any contaminating cells. The quantification of IFN-7 released by CAR T cells in the supernatant was performed using the DuoSet® ELISA kit for mouse IFN-γ (R&D systems, DY485). Nunc MaxiSorp flat-bottom plates (Thermo Fisher Scientific, 44-2404-21) were employed for the assay, conducted on a Tecan Infinite 200 Pro machine according to the manufacturer's instructions. To maintain the assay within the linear range of the kit, the supernatant was initially diluted at 1:10 in reagent diluent. Subsequently, a minimum of six serial 4-fold dilutions were executed. For each plate, at least one standard curve was generated, and the entire experiment included at least two standard curves, constructed using standard solutions supplied by the manufacturer. The substrate solution used was 1-Step™ Ultra TMB-ELISA (Thermo Fisher Scientific, 34028), and the stop solution employed was 2N sulfuric acid (VWR, BDH7500-1). Bovine serum albumin (BSA; Sigma, A8022-500G) was prepared as a sterile-filtered 5% stock in PBS (Corning, 21-031-CV).

Mouse maintenance and studies. All animal studies were conducted in compliance with approved protocols from the MIT Committee on Animal Care (protocol number 0521-028-24). The mouse strains utilized in this study included C57BL/6 (Jackson Laboratory) and NOD/SCID/IL-2Rg^{-/-} (NSG; Jackson Laboratory). Immunocompetent C57BL/6 mice underwent sub-lethal irradiation (1×5 Gy) immediately before the transplantation of B-ALL cells. The B-ALL cells for transplantation were suspended in saline and administered via tail vein injection using 29-gauge syringes. Subsequently, mice received CAR T cells via tail vein injection, employing 29-gauge syringes, two or three days later, as indicated.

Tracer binding experiments in vitro. To assess the binding efficiency of the CD19 tracer to CAR T cells in vitro, 1×10⁵ CAR T cells were subjected to incubation with varying concentrations (ranging from 0 nM to 100 nM) of the CD19 tracer, which was either conjugated to Alexa647, DFO, or DFO along with non-radioactive Zirconium (Zr). The binding of hCD19 tracers coupled to DFO or DFO and cold Zr was determined using anti-FLAG tag antibody APC (Biolegend, #637308). Triplicate analyses were conducted for all tracer concentrations.

Tracer experiments in vivo. In vivo tracer binding experiments were conducted using C57BL/6 mice. The mice underwent sub-lethal irradiation (1×5 Gy) and were transplanted with 3×10⁶ B-ALL cells via tail vein. Subsequently, 10×10⁶ CAR T cells were injected via tail vein three days post-transplantation. After two days, mice received an injection of 5 μg Alexa647-labeled CD19 tracer via tail vein. Five hours post-tracer injection, mice were sacrificed, and blood, spleen, and bone marrow were collected for flow cytometric analysis. Flow cytometry was utilized to determine the percentage of CD19 CAR T cells or control CAR T cells (GFP⁺) bound by the CD19 Alexa647-labeled tracer.

In survival experiments, C57BL/6 mice underwent sub-lethal irradiation (1×5 Gy) and were transplanted with 3×10⁶ B-ALL cells via tail vein. Two days post-transplantation, 10×10⁶ CAR T cells were injected via tail vein. Three days later, mice received an injection of 5 μg DFO-PEG20-labeled CD19 tracer coupled to cold Zr. Cold Zr labeling was achieved by incubating the CD19 tracer with ZrCl4 solution (1:3 molar ratio CD19 tracer to ZrCl4) for 1 hour before usage. Mice were sacrificed at humane endpoints as defined by the CAC guidelines.

Installation of ⁸⁹Zr isotope to the CD19 tracer and PET imaging. A working stock of ⁸⁹Zr in 1M Oxalic Acid was created by neutralizing the 1M Oxalic Acid with a half volume of 2M Na₂CO₃ followed by 0.5M HEPES buffer. Subsequently, an appropriate amount of the working stock was measured and mixed with the DFO-labeled CD19 tracer, incubated on a shaker at room temperature for 1 hour or more. The ⁸⁹Zr-CD19 mixture was then filtered through a PD-10 column (Cytiva 17-0851-01) to remove unbound isotope, and radiolabeled fractions were collected and pooled for injection. PET images were acquired using a Sofie G8 PET machine. Static scans were conducted with a 10-minute PET acquisition time followed by a 2-minute pCT. Images were reconstructed using the internal reconstruction method (MLEM 3D) and output into a DICOM stack.

For PET imaging experiments, C57BL/6 mice were sub-lethally irradiated (1×5 Gy) and transplanted with 3×10⁶ B-ALL cells via tail vein. Three days later, lox 10⁶ CAR T cells were injected via tail vein. After an additional two days, mice were injected with 5-10 g CD19 radiolabeled tracer coupled with ⁸⁹Zr via tail vein. To determine the tracer's half-life in mouse blood, samples were collected at 5 min, 10 min, 15 min, 30 min, 1.5 hours, 3 hours, 4 hours, 6 hours, 16 hours, 24 hours, and 48 hours post-injection. Mice were imaged starting 24 hours after radiolabeled tracer injection and monitored regularly via PET imaging until reaching moribund status. Upon reaching this point, mice were sacrificed, and their organs were harvested and analyzed for radioactivity to ascertain the biodistribution of the CD19 radiolabeled tracer.

All statistical analyses were conducted using GraphPad Prism 10 (GraphPad Software Inc). The specific statistical tests performed for each analysis are outlined in the figure legends. Differences were considered statistically significant for P-values ≤0.05.

CAR imaging in syngeneic solid tumor mouse model. An autochthonous pancreatic mouse model was established by orthotopically engrafting 5×10⁵ murine pancreatic cancer cells (LSL-Kras^{G12D/+}. LSL-hp53^{R172H/+}; Pdx1-Cre) ectopically expressing human CD19. Four days post-engraftment of the cancer cells, 5×10⁵ CD19 CAR T cells were injected intraperitoneally into the mice. Next, mice were injected with a radiolabeled tracer containing the CD19 ectodomain and ⁸⁹Zr and subjected to PET imaging 1-, 10-, and 20-days post injection (radiolabeled tracer was injected each day before imaging).

PET-CT studies, and ex vivo biodistribution studies are described in Rashidian et al., J. Exp. Med. 214(8):2243-2255 (2017). In brief, mice were anesthetized and imaged by PET-CT using a G8 PETCT small-animal scanner (PerkinElmer). Peak sensitivity of this instrument accounts for >14% of positron emission, with a mean resolution of 1.4 mm. Each PET acquisition was 10 min long, followed by a 1.5 min CT scan. Images were processed using automatic image reconstruction software provided by the manufacturer. Images are further analyzed and quantified via VivoQuant™ software to determine percentage of injected dose per gram (% ID/g) and standard uptake values (SUVs) for all relevant organs (Rashidian et al., Proc. Natl. Acad. Sci. U.S.A. 112(19):6146-51 (2015); Rashidian et al., J. Exp. Med. 214(8):2243-2255 (2017)). The PET and CT scan were overlaid to guide generation of 3D regions of interest (ROIs) that represent a certain organ within the mouse. The ROI results were exported as a table containing statistical information including mean PET signal or variation. These values were compared across each time point and evaluated by t-test for significant differences between the PET signal in the control and experimental mice. For ex vivo biodistribution studies, mice were administered i.v. with ~100 μCi of radiolabeled tracer (~5 g), euthanized 4 hours later, perfused with PBS (20 mL), and dissected. Tissues, blood, and urine were collected, and their wet weight determined. Radioactivity was measured with a Perkin-Elmer Wallac Wizard 3″ 1480 Automatic Gamma Counter. Statistical analyses were performed via the GraphPad Prism software and data expressed as (% ID/g) (excretion subtracted).

Blood half-life measurements of the radiolabeled tracers. Mice were i.v. administered ~100 μCi of each of the radiolabeled tracers (~5 g). Blood samples were obtained by retroorbital puncture using tared, heparinized capillary tubes. The samples and capillaries were weighed, and radioactivity was measured using a Perkin-Elmer Wallac Wizard 3″ 1480 Automatic Gamma Counter. Values, expressed as % ID/g of tissue, were fit (least squares) to a two-compartment biexponential decay model using GraphPad Prism software.

### Example 2: Developing CD19 and BCMA Ectodomain Radiolabeled Tracers

To show that cancer ectodomains can be used as radiolabeled tracers, cancer antigens were site-specifically modified with sortase to incorporate radioactive isotopes for PET imaging. Sortase radiolabeling methods are site-specific, stoichiometric, rapid, robust, reproducible, and produce high yields. Sortase has been used to develop several radiolabeled proteins, including nanobodies against CD11b, Class II MHC, PD-L1, and CD8 to image immune responses and observed no affect to their binding efficiency (Rashidian et al., Proc. Natl. Acad. Sci. U.S.A 112(19):6146-51 (2015); Rashidian et al., J. Exp. Med. 214(8):2243-2255 (2017); Rashidian et al., Proc. Natl. Acad. Sci. U.S.A 116(34):16971-16980 (2019); Rashidian et al., Acs Cent. Sci. 1(3):142-147 (2015); Ingram et al., Nat. Commun. 8(1):647 (2017)).

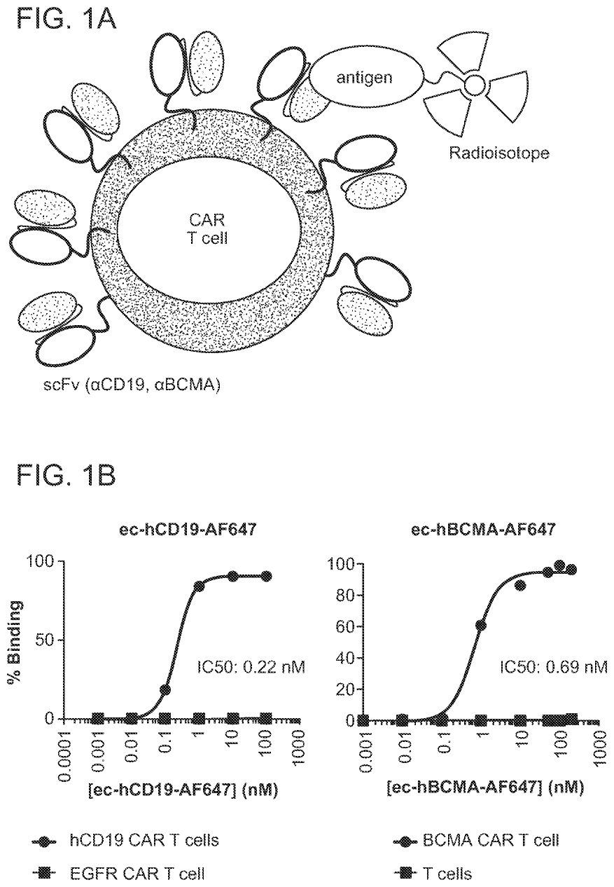

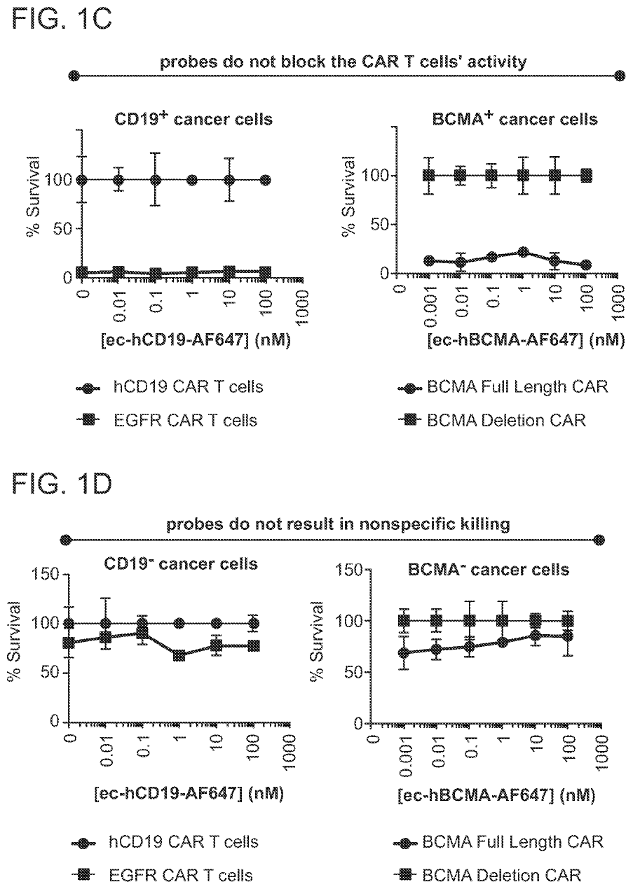

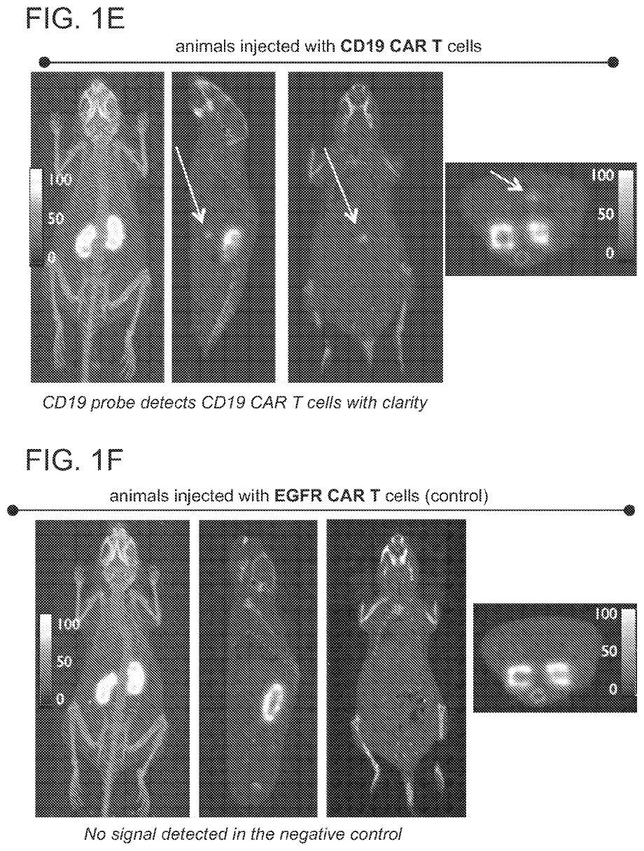

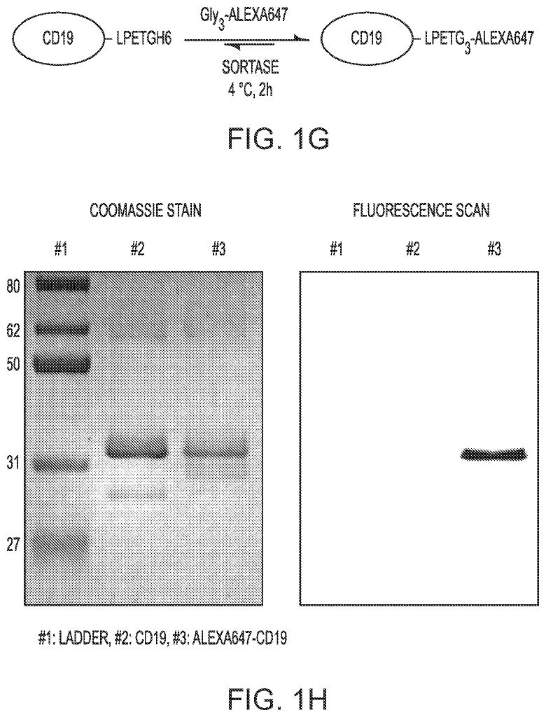

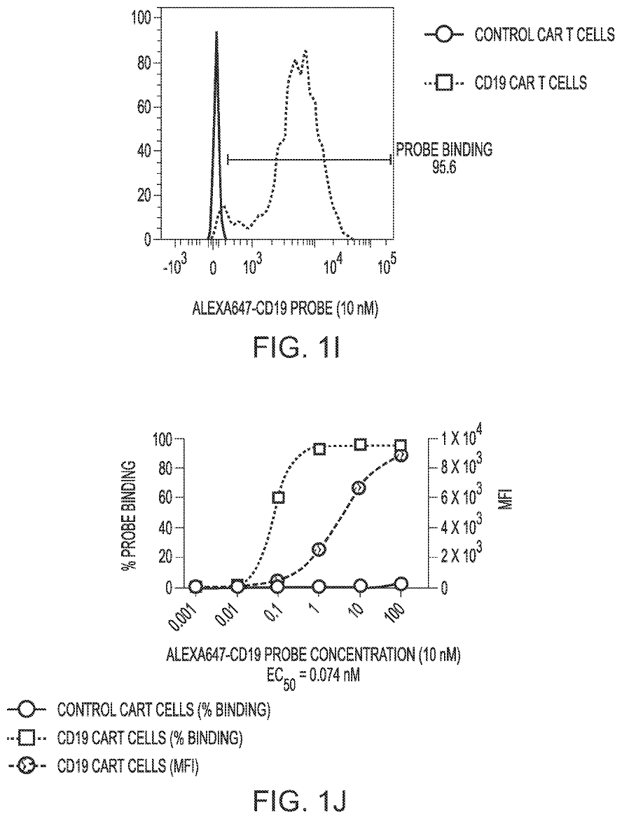

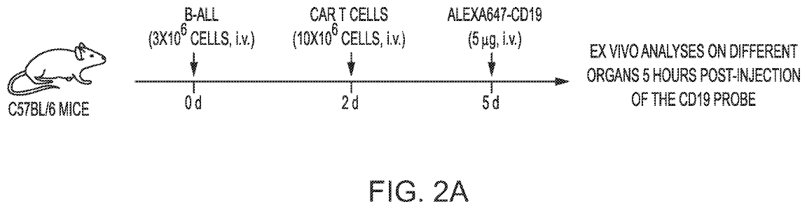

CD19 is a 58 kilodalton type I transmembrane glycoprotein expressed in almost all human B lineage cells, except plasma cells (Fearon and Carroll, Annu. Rev. Immunol. 18:393-422 (2000)). CD19 is a biomarker for B lymphocyte development, lymphoma diagnosis, and is widely used as a target for CAR T leukemia immunotherapies (Sato et al., Proc. Natl. Acad. Sci. U.S.A 92(25):11558-11562 (1995); Locke et al., Mol. Ther. 25(1):285-295 (2017)). A radiolabeled tracer containing the ectodomain of human CD19 (ec-hCD19) has a mass of approximately 32 kDa and is an ideal imaging agent to target and monitor CD19 CAR T. Accordingly, Alexa647-labeled and radiolabeled tracers containing ec-hCD19, a C-terminal FLAG purification tag (FLAG-tag; DYKDDDDK (SEQ ID NO: 43)), and the sortase recognition motif LPETG (SEQ ID NO: 47) for site-specific modifications (e.g., radioactive tracer connections) were created to evaluate in vivo metabolic fate and pharmacokinetic properties. The sortase enzyme, a bacterial transpeptidase, recognizes the LPETG motif (SEQ ID NO: 47), catalyzing the cleavage of the T-G bond to form an acyl intermediate. Upon addition of a “Gly3-R” substrate, this intermediate evolves into the “protein-LPET-Gly3-R” product, wherein R can represent various biomolecules of interest (FIG. 1G). Purification of the CD19, Alexa647-labeled tracer was accomplished through immobilized metal chelate affinity chromatograph (IMAC) column chromatography followed by size-exclusion chromatography, resulting in a yield of approximately 10 mg per liter of cultured cells. The tracer was further characterized via SDS-PAGE analysis (FIG. 1H).

BCMA, or tumor necrosis factor receptor superfamily member 17 (TNFRSF17), is a cell surface receptor which recognizes B-cell activating factor (BAFF) (O'Connor et al., J. Exp. Med. 199(1):91-98 (2004); Gross et al., Nature 404(6781):995-999 (2000), Schiemann et al., Science 293(5537):2111-2114 (2001)). Preferentially expressed in mature B lymphocytes, BCMA is involved in diseases such as leukemia, lymphomas, and multiple myeloma, and thus has been used as a target in both preclinical and clinical studies (Raje et al., N. Engl. J. Med. 380(18):1726-1737 (2019); Chang et al., Chang Gung Med. J. 29(5):532-537 (2006); Huang et al., Proc. Natl. Acad. Sci. U.S.A 110(27):10928-10933 (2013); Hymowitz et al., J. Biol. Chem. 280(8):7218-7227 (2005); Laabi et al., Embo J. 11(11):3897-3904 (1992); Mahmoodi et al., Cancer Genet. Cytogenet. 154(2):160-162 (2004); Lee et al., Leukemia 35(1):255-258 (2021)). BCMA CAR T cells have been extensively used for MM with an overall response rate of 90% (Raje et al., N. Engl. J. Med. 380(18):1726-1737 (2019); D'Agostino and Raje, Leukemia 34(1):21-34 (2020)). However, the responses are frequently not durable and most of the patients suffer a relapse, due to low persistence of CAR cells (Raje et al., N. Engl. J. Med. 380(18):1726-1737 (2019); D'Agostino and Raje, Leukemia 34(1):21-34 (2020); Munshi et al., N. Engl. J. Med. 384(8):705-716 (2021)).

Patients with extramedullary and medullary lesions particularly have a lower rate of response and higher relapse rate (Raje et al., N. Engl. J. Med. 380(18):1726-1737 (2019); D'Agostino and Raje, Leukemia 34(1):21-34 (2020)). Imaging BCMA CAR T cells enables the dynamics of the cells post-infusion to be imaged. The imaging of BCMA CAR T cells enables the understanding of issues such as where the BCMA CAR T cells home after infusion, how well the cells can infiltrate into the bone marrow and for how long persist there, and the CAR T cell population in different organs changes over time. Significant clinical understanding to therapy response may be gained if patients receiving BCMA CAR T cells can be imaged once in about 3-4 months with a BCMA PET radiolabeled tracer for about 1-2 years post-infusion of cells. Similar to CD19 CAR imaging, the N-terminal ectodomain of BCMA (ec-hBCMA) is about 7 kDa, and is an ideal radiolabeled tracer to image BCMA CAR T cells.

### Example 3: Effects of BCMA and CD19 Ectodomain-Containing Radiolabeled Tracers

The effects of ec-hBCMA and ec-hCD19 radiolabeled tracers on the activity of CAR T cells were determined. Radiolabeled tracers containing a cancer antigen ectodomain have the potential to block or affect the activity of CD19 or BCMA CAR T cells. Ideally, a PET radiolabeled tracer should not have any significant effect on the target cell to which it binds. Therefore, a killing assay using CAR T cells and antigen-positive tumor cells was performed for the CD19 CAR T cells and BCMA CAR T cells in the presence of varying concentrations of the radiolabeled tracer containing either ec-hCD19 or ec-hBCMA, along with controls using irrelevant CAR cells and antigen-negative tumors. Due to the dynamic nature of the CAR interaction with the antigen on tumor cells, it is not expected that the radiolabeled tracers will have significant effects on the activity of the CAR cells, in vitro or in vivo. Most multiple-myeloma patients have high levels of soluble BCMA in the circulation, due to y-secretase-dependent shedding (Munshi et al., N. Engl. J. Med. 384(8):705-716 (2021)). However, and despite presence of these soluble BCMA in the circulation, patients receiving the BCMA CAR T cells have shown remarkable responses.

Robust sortase-mediated methods to site-specifically radiolabel proteins with different radioactive isotopes, including the clinically used isotopes ¹⁸F (t_{1/2}=110 min), ⁶⁴Cu (t_{1/2}=12.7 h) and ⁸⁹Zr (t_{1/2}=3.3 days) have been developed (Rashidian et al., Proc. Natl. Acad. Sci. U.S.A 112(19):6146-51 (2015); Rashidian et al., Acs Cent. Sci. 1(3):142-147 (2015); Rashidian et al., J. Exp. Med. 214(8):2243-2255 (2017)). Tracers radiolabeled with ⁶⁴Cu and ⁸⁹Zr allow for imaging up to at least 24 hours after radiolabeled tracer administration. Initial studies were performed in healthy mice to study which isotope yielded less non-specific background signal. For example, to establish the approach, the ec-hCD19 tracer was labeled with i) NOTA or ii) DFO using sortase. Next, they were labeled with ⁶⁴Cu and ⁸⁹Zr, respectively, and healthy mice were injected with ~50 μCi of each of the radiolabeled tracers (~5 μg), followed by PET imaging after 24 hours.

PET images showed high liver background for the ⁶⁴Cu-CD19 radiolabeled tracer, but no significant liver uptake was observed in the ⁸⁹Zr-CD19 radiolabeled tracer injected mice. Both radiolabeled tracers were cleared via kidneys, and no other organs showed significant uptake. Similar studies were performed for BCMA radiolabeled tracer to determine which isotope is best to pursue the studies. To ensure that the radiolabeling does not compromise the functionality of the radiolabeled tracers and to study their stability, non-radioactive copper and zirconium were installed onto the tracers, chelation was confirmed via LC-MS analyses, and tested by flow cytometry using the metal-labeled (Cu or Zr)— radiolabeled tracers on CD19 and BCMA CAR T cells. Both radiolabeled tracers were engineered with a FLAG-tag at their C-termini (an 8 amino acid motif: DYKDDDDK (SEQ ID NO: 43)), allowing the use of an anti-FLAG antibody as secondary staining for flow cytometric analyses. The metal-labeled (Cu or Zr)-CD19 radiolabeled tracer remained fully functional and stained 100% of CD19 CAR T cells at low concentration range (5 nM) similar to non-metal labeled tracer.

Tracers were bacterially expressed, fluorophore-labeled via sortase, and characterized via SDS-PAGE and LC-MS analyses. The labeled tracers are illustrated in FIG. 1A. The tracers stained CD19 and BCMA CAR T cells with an EC50 of 0.22 nM and 0.69 nM, respectively, with high specificity (FIG. 1B). Neither tracer bound to any human PBMC cells, even when used at a concentration of 100 nM. MC38-tumor bearing C57BL/6 mice were injected with AF647-labeled tracers (2 nmol per mouse; n=3) and lymphoid organs and tumors were excised and analyzed after 3 h. No non-specific staining was observed. Killing assays using CAR T cells and tumor cells were performed in the presence of varying concentrations of tracers. Impressively, the tracers caused no blocking of CAR T cell activity (FIG. 1C), and no killing of antigen-negative tumor cells was observed (FIG. 1D). To assess the tracer in vivo behavior, radiolabeled tracer containing ec-hCD19 labeled with 8⁹Zr was injected into mice bearing orthotopic hCD19⁺-PDAC tumors that received 5×10⁶ hCD19 CAR T cells or irrelevant EGFR CAR T cells and imaged after 24 h. PET imaging acquired 24 hours post-injection revealed radiolabeled tracer accumulation, indicating the presence of CD19 CAR T, in the tumor, but not in the control group (FIG. 1E-FIG. 1F).

### Example 4: CD19 Alexa647-Labeled Tracer Results in High Affinity, High Specificity CD19 CAR T Cell Binding

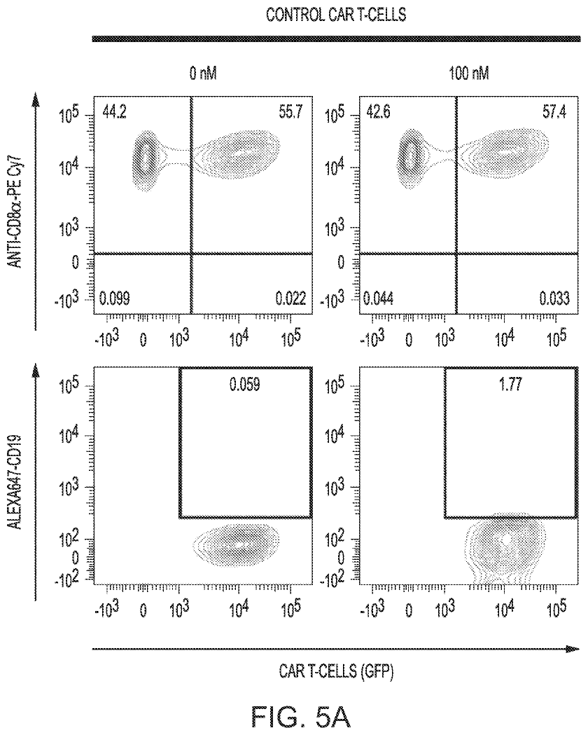

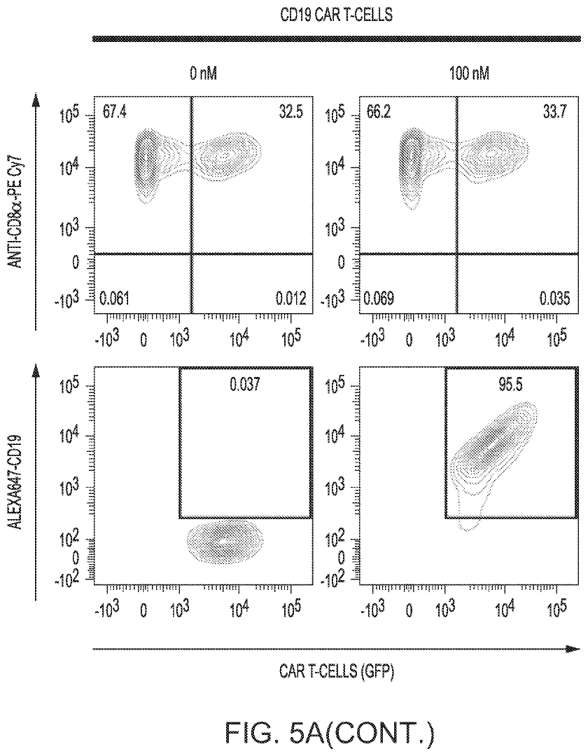

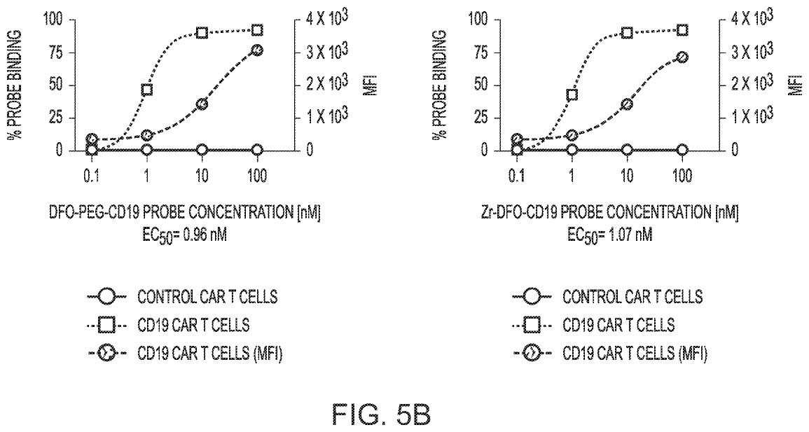

CD19, Alexa647-labeled tracer-CAR T cell binding was evaluated using flow cytometry. Murine T cells expressing a CAR targeting human CD19 or control CAR targeting epidermal growth factor receptor variant III (EGFRvIII) were utilized. CAR T cells were incubated for 30 minutes with 10 nM of CD19 tracer, site-specifically labeled with Alexa647 (Alexa647-CD19 tracer) through a sortase reaction (FIG. 1H). This process resulted in strong staining of CD19 CAR T cells, as demonstrated in FIG. 1I. Conversely, the control CAR T cells remained unstained (FIG. 1I and FIG. 5A). In FIG. 5A, one hundred thousand CAR T cells were incubated for 30 minutes with various concentrations of Alexa647-, DFO-PEG-, or Zr-DFO-PEG-labeled CD19 tracer (ranging from 100 nM to 0 nM in 10-fold dilutions) and subsequently stained for CD8a (PE-Cy7), CD8a⁺ and GFP⁺ CAR T-cells were gated for subsequent analysis. The gating strategy for flow cytometric analysis of Alexa647-CD19 tracer binding to control (EGFRvIII) and CD19 CAR T cells is displayed for the 0 and 100 nM concentrations.

To determine the half-maximal concentration (EC₅₀) required for labeling CD19 CAR T cells, 100,000 CD19 CAR T cells or control EGFRvIII CAR T cells were incubated for 30 minutes with decreasing amounts of Alexa647-labeled CD19 tracer, ranging from 100 nM to 0 nM in 10-fold dilutions (FIG. 1J). The results demonstrated dose-dependent fluorescent labeling of CD19 CAR T cells at concentrations exceeding 10 μM, with a picomolar range EC₅₀ (EC₅₀=0.07 nM). Typically, for in vivo PET imaging, the required blood concentration of an imaging tracer is in the low single-digit nM range (Mayer et al., J. Nucl. Med. 58(4):538-546 (2017), Mayer and Gambhir, J. Nucl. Med. 59(8):1174-1182 (2018)).

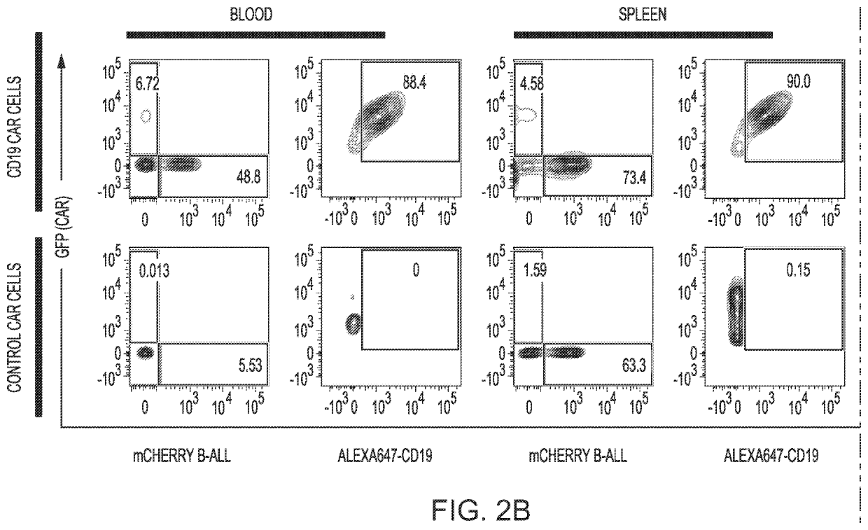

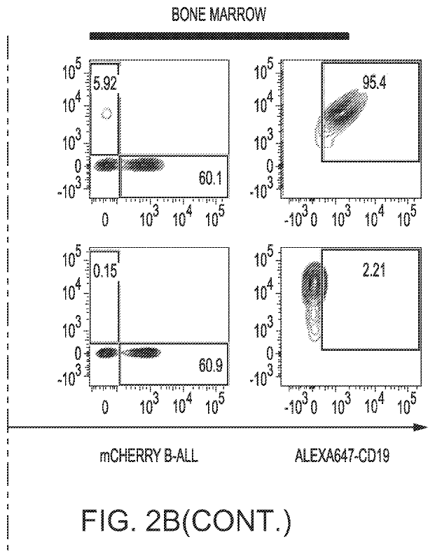

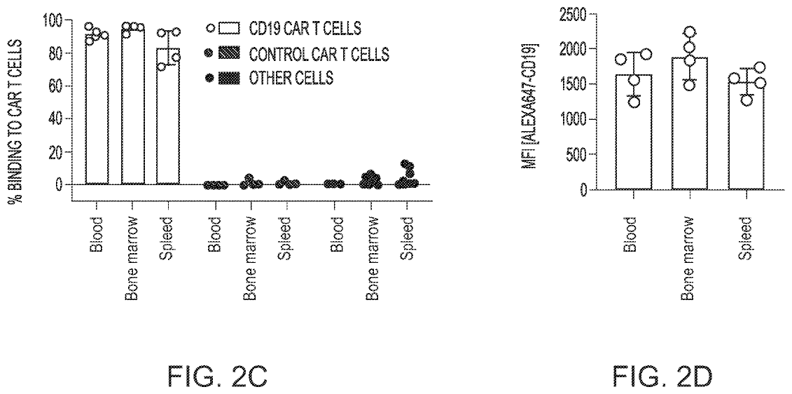

Next the CD19 tracer's ability to bind to CD19 CAR T cells was assessed in vivo. A transplantable, immunocompetent mouse model of BCR-ABL+ B-cell acute lymphoblastic leukemia (B-ALL) was employed. This mouse model serves as a preclinical model for the most common type of leukemia treated with CAR T cell therapy (Dorr et al., Proc. Natl. Acad. Sci. U.S.A. 111(37):13343-8 (2014)). The B-ALL cells were genetically engineered to express the ectodomain of human CD19 on their cell surfaces (hCD19⁺ B-ALL). Mice transplanted with B-ALL cells develop an aggressive form of leukemia and carry a leukemic burden in their blood, bone marrow, and spleen (Williams et al., Proc. Natl. Acad. Sci. U.S.A 103(17):6688-93 (2006)). C57BL/6 mice that had previously received sub-lethal irradiation (1×5 Gy) were transplated with hCD19⁺ B-ALL cells. Two days post transplantation, anti-human CD19 CAR T cells or control (anti-EGFRvIII or anti-human CD20) CAR T cells were injected into the tail vein. Three days post-CAR T cell injection, a tail vein injection of 5 g of Alexa647-labeled CD19 tracer was administered. Five hours post-tracer injection, blood, bone marrow, and spleen samples were collected for flow cytometric analysis (FIG. 2A). Gating on CAR T cells (GFP⁺) in each respective organ revealed robust Alexa647 labeling in CD19 CAR T cells (91.5% in blood, 94.5% in bone marrow, and 83.3% in the spleen) but undetectable staining in either control CAR T cells or non-CAR T cell populations (FIG. 2B-FIG. 2C). Moreover, the MFI of Alexa647-CD19 tracer binding to CD19 CAR T cells was consistently high in all analyzed organs (FIG. 2D). Collectively, these results affirm the CD19 tracer's high-affinity and specific binding to the CD19 CAR T cells, both in vitro and in vivo.

### Example 5: CD19 Tracer does not Impact Functionality of CAR T Cells

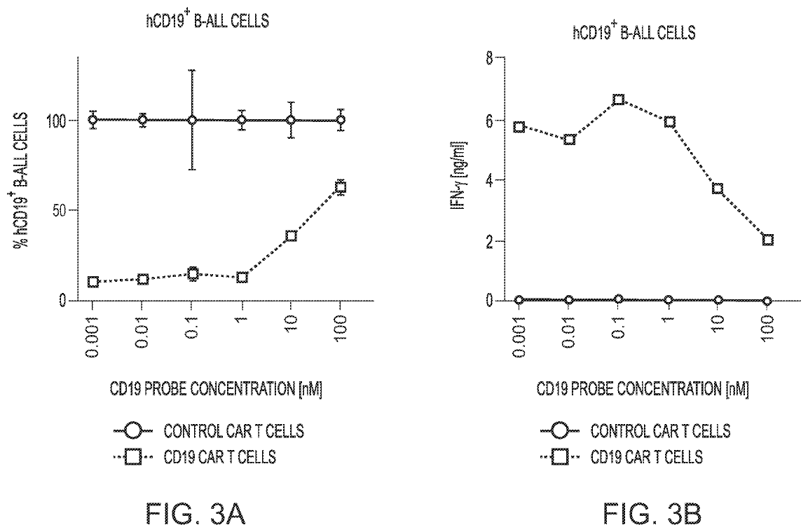

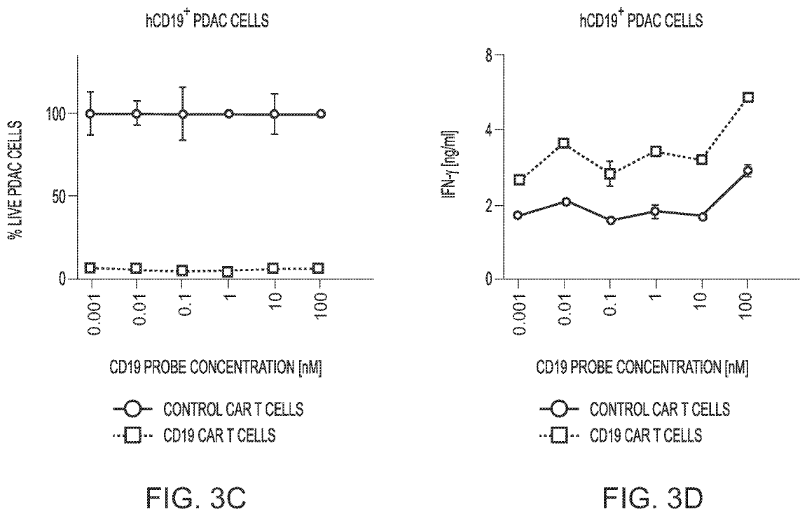

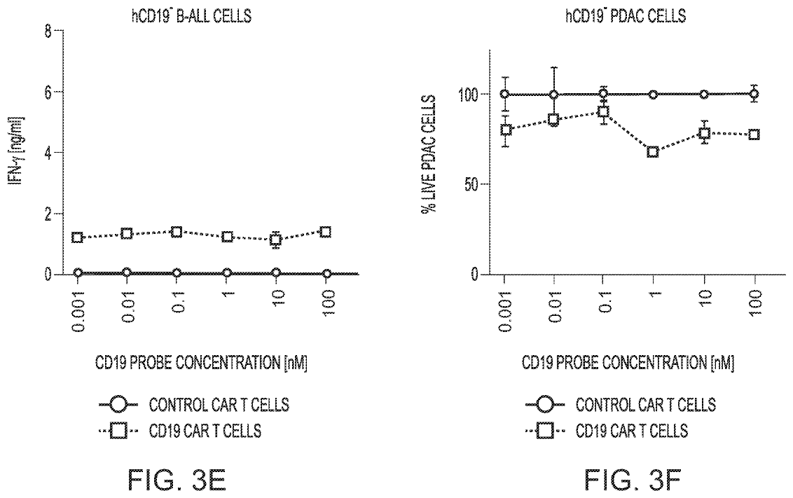

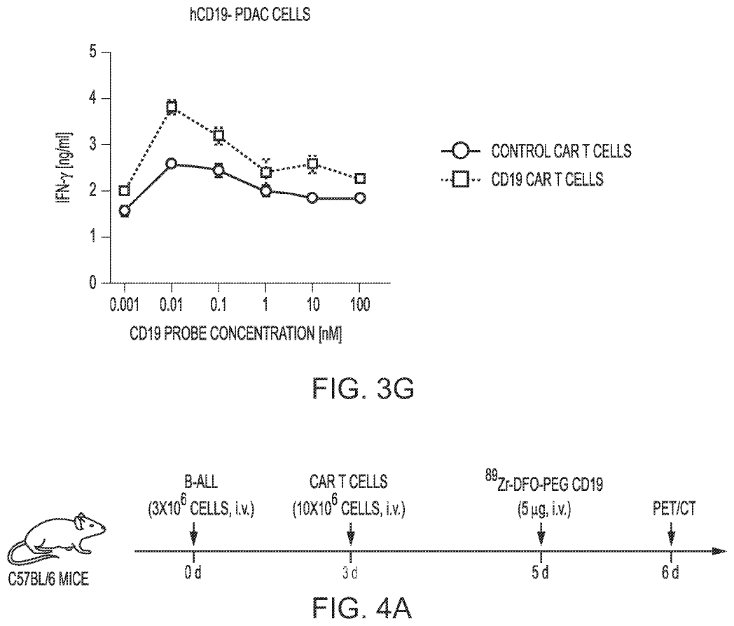

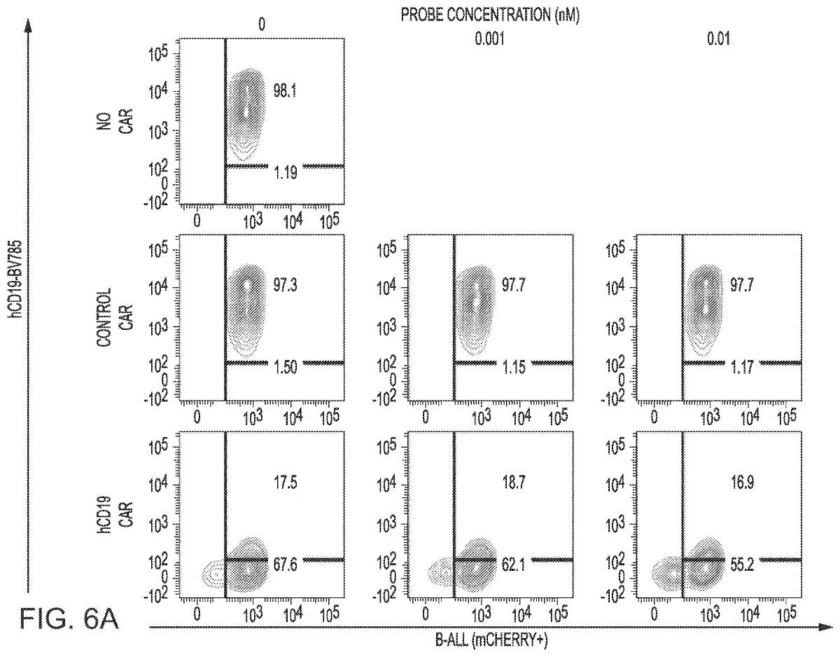

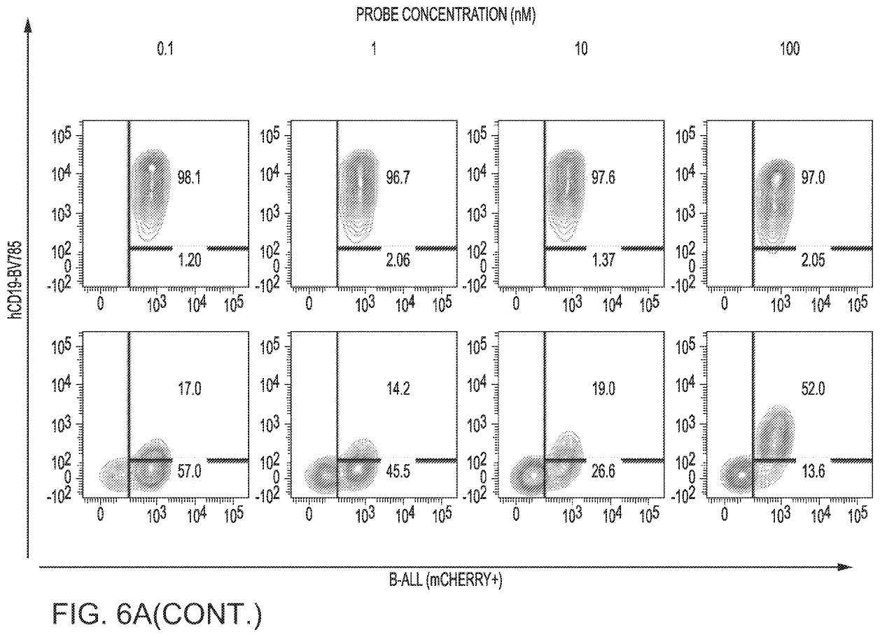

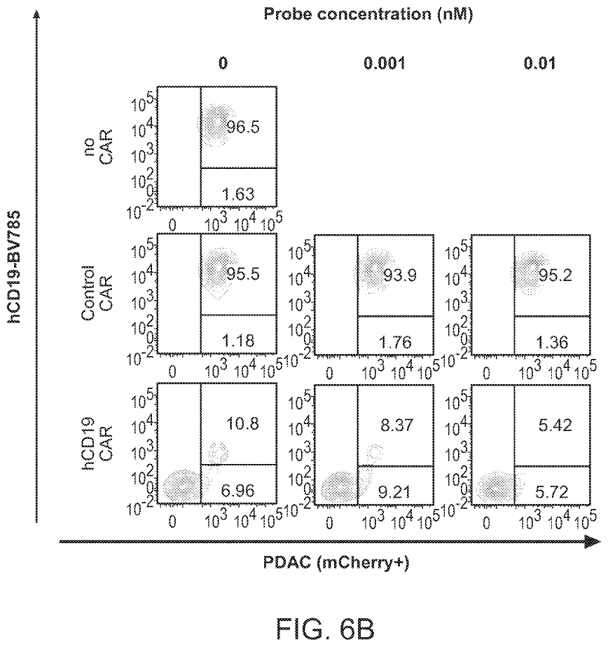

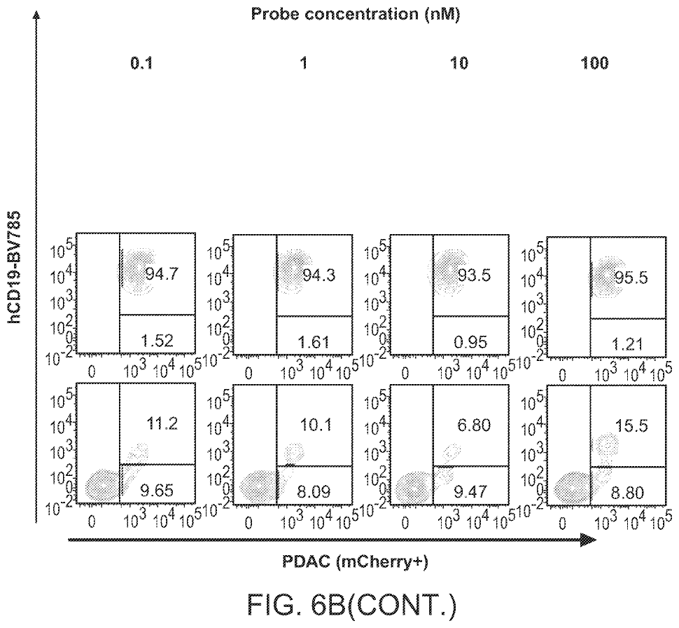

The next objective was to investigate the impact of the CD19 tracer on CD19 CAR T cell functionality. First, it was assessed whether the interaction between the CD19 tracer and CD19 CAR on T cells impeded CAR T cell killing activity in vitro. To do this, CAR T cells were co-cultured with either murine B-ALL cells (hCD19⁺ B-ALL) or murine pancreatic cancer cells engineered to express the ectodomain of human CD19 (hCD19⁺ PDAC). Human CD19+B-ALL cells are typically less susceptible to CAR T cell-mediated killing in vitro due to the rapid endocytosis of the target antigen, thus hCD19⁺ PDAC cells were included as an additional experimental condition. B-ALL and PDAC cells were incubated with either CD19 or control CAR T cells at an E:T ratio of 10:1 with increasing concentrations of the Alexa647-labeled CD19 tracer (0 nM to 100 nM). After 24 hours, endpoint data was evaluated by flow cytometry. Given the decreased susceptibility to killing observed in hCD19⁺ B-ALL cells in vitro, surface levels of CD19 on B-ALL cells as well as release of IFN-γ by CAR T cells in the assay were assessed as indicators of CAR T cell function (FIG. 3A, FIG. 3B, FIG. 3E, and FIG. 6A). For hCD19⁺ PDAC cells, viability and IFN-γ release were evaluated for CAR T cell function (FIG. 3C, FIG. 3D, FIG. 3F, FIG. FIG. 3G, and FIG. 6B).

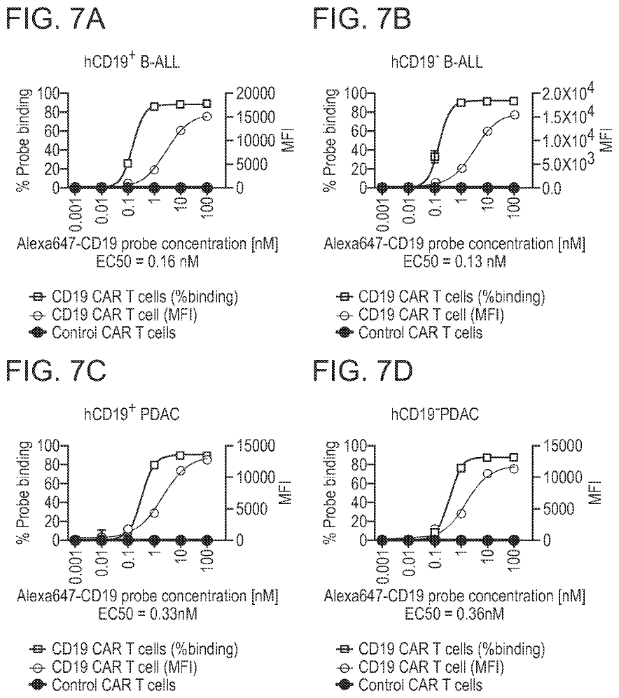

The Alexa647-labeled CD19 tracer was not removed during the killing assays and remained bound to CAR T cells in a dose-dependent manner at the time of analysis (FIG. 7A-FIG. 7D); the CD19 CAR T cells were murine T cells transduced with a CAR construct containing an anti-human CD19 ectodomain and a murine CD28-CD3ζ intracellular domains. The presence of the CD19 tracer at concentrations of 1 nM or lower did not affect antigen loss on B-ALL cells (FIG. 3A). An increase in antigen retention on B-ALL cells was only observed when the tracer concentration reached 10 nM or greater (FIG. 3A). Furthermore, when evaluating the levels of IFN-γ released by CD19 CAR T cells or control CAR T cells, a dose-dependent reduction in IFN-γ production from CD19 CAR T cells was observed when they were co-cultured with hCD19⁺ B-ALL cells and exposed to tracer concentrations of 10 nM or greater (FIG. 3B). The Alexa647-labeled CD19 tracer did not hinder the killing of hCD19⁻ PDAC cells, even at tracer concentrations as high as 100 nM (FIG. 3C). Furthermore, CD19 CAR T cells did not exhibit reduced IFN-γ release, even when the tracer concentration reached 100 nM in the presence of hCD19⁺ PDAC cells (FIG. 3D). These results indicate that the tracer does not hinder CAR T cell killing activity. Without being bound by theory, the dominance of membrane-bound CD19 antigen, facilitated by synapse formation between CAR and tumor cells, likely leads to higher avidity interactions with CAR molecules, preventing interference from soluble CD19 antigen binding.

Non-specific killing of antigen-negative B-ALL or PDAC cells by CD19 tracer bound CD19 CAR T cells was assessed. Killing assays were performed in which antigen-negative B-ALL or PDAC cells were combined with CD19 CAR T and control CAR T cells, followed by the addition of increasing concentrations of Alexa647-labeled CD19 tracer. When CD19 CAR T cells were co-cultured with antigen-negative B-ALL cells, there was no significant release of IFN-γ, and the level of IFN-γ released remained consistent regardless of the quantity of Alexa647-labeled CD19 tracer added to the killing assay (FIG. 3E). This result demonstrated that only interaction with antigen-positive B-ALL cells could stimulate IFN-7 release from the CD19 CAR T cells, while the interaction with the CD19 tracer did not have this effect. Similarly, when CD19 CAR T cells were mixed with antigen-negative PDAC cells and varying amounts of the tracer, no dose-dependent killing (FIG. 3F) or IFN-γ release (FIG. 3G) was observed.

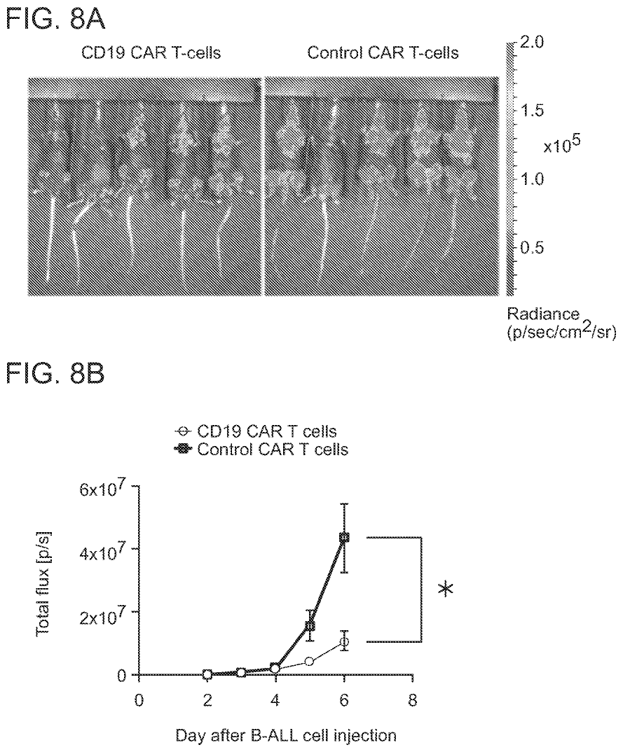

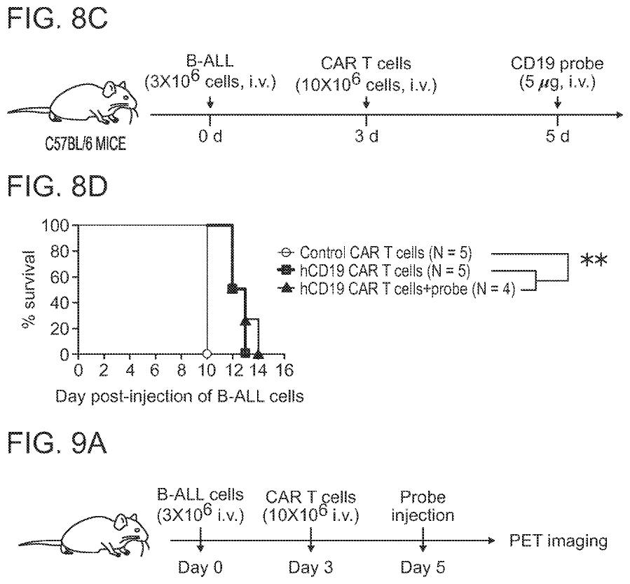

To replicate the complex conditions encountered by CAR T cells in vivo, it was next investigated whether administration of the CD19 tracer impacts CAR T cell function in a syngeneic model. It was first established that in vivo hCD19⁺ B-ALL tumor burden is significantly reduced with treatment with CD19 CAR T cells but not control antigen (EGFRvIII)-targeting CAR T cells (FIG. 8A-FIG. 8B). Next, mice that had been previously sub-lethally irradiated (1×5 Gy) were transplanted with 3×10⁶ hCD19⁺ B-ALL cells and, three days later, administered CAR T cells targeting human CD19 or the control antigen (EGFRvIII). While radiation is not necessary for tumor cell engraftment in this syngeneic model, it mirrors the pretreatment priming received by patients prior to CAR-T engraftment. Following two days of CAR T cell treatment, 5 μg of the CD19 tracer were injected into the mice. This CD19 tracer was labeled with the chelator DFO and loaded with cold Zirconium (Zr) ion, mirroring the PET tracer that will be used for PET imaging (FIG. 8C). Treatment with CD19 CAR T cells significantly prolonged the survival of leukemic mice compared to those treated with control CAR T cells (FIG. 8D). Importantly, there was no disparity in survival between mice treated with CD19 CAR T cells with or without the CD19 tracer. Therefore, these results indicate that the CD19 tracer does not interfere with the functional activity of CD19 CAR T cells in vivo.

### Example 6: Radiolabeled CD19 Tracer Detects CD19 CAR T Cells In Vivo with High Specificity

First, it was assessed whether the inclusion of the chelator DFO and zirconium ions would impact the binding characteristics of the CD19 tracer to CD19 CAR T cells. ⁸⁹Zr was used as the PET isotope because of its broad clinical applicability and a half-life of approximately 3.3 days, allowing for imaging several hours after the tracer injection. CD19 CAR T cells and control CAR T cells underwent incubation with the CD19 tracer, which had been site-specifically labeled with DFO chelator using a sortase reaction. Both CD19 tracer labeled with DFO alone (DFO-CD19) and CD19 labeled with DFO installed with Zirconium ion (Zr-DFO-CD19) was assessed. The concentrations ranged from 0 nM to 100 nM. For detection of the tracer, an anti-FLAG secondary antibody was used. The results demonstrated that both DFO-CD19 and Zr-DFO-CD19 (FIG. 5B) displayed specific binding to CD19 CAR T cells, with no binding observed on the control CAR T cells. Additionally, both constructs exhibited a high binding affinity to CD19 CAR T cells with low nM affinity, which closely paralleled the findings obtained with the Alexa647-CD19 tracer. Therefore, the site-specific introduction of DFO and zirconium ions did not impact the tracer's binding to CD19 CAR T cells.

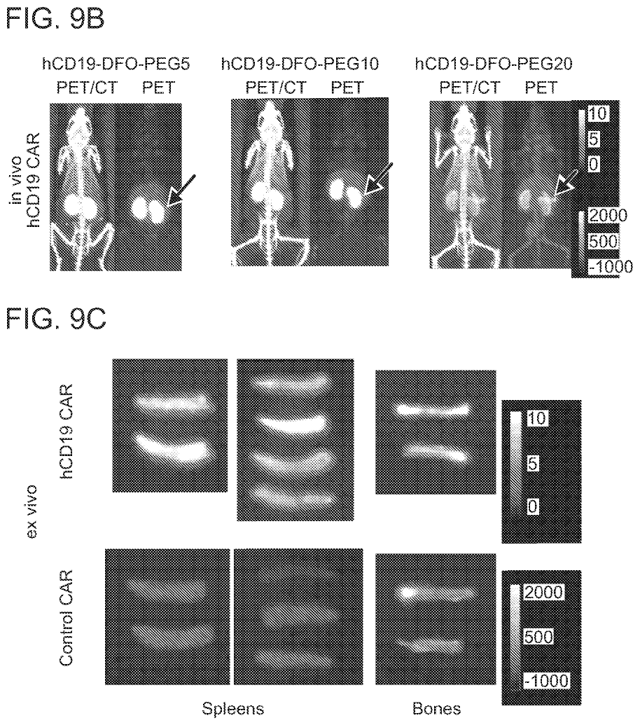

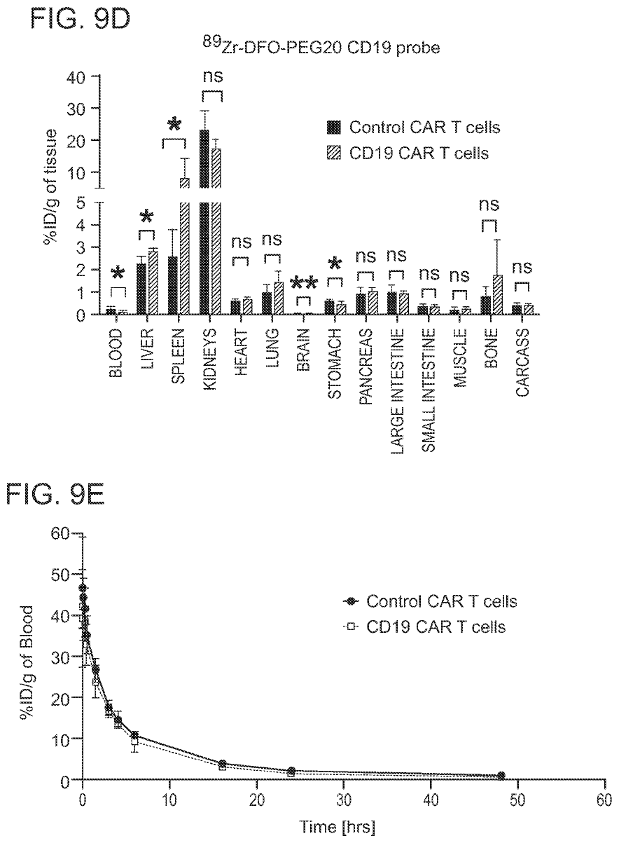

Next, PET imaging to visualize CD19 CAR T cells was performed. To facilitate this, the DFO-CD19 tracer was conjugated with radioactive ⁸⁹Zr ion and administered to C57BL/6 mice that had previously received transplants of B-ALL cells and been treated with either control or CD19 CAR T cells (FIG. 9A). For optimization of in vivo splenic visualization, a comparison of non-specific kidney retention of the CD19 tracer when linked to varying lengths of polyethylene glycol (PEG) was conducted. PEGylation of the CD19 tracer resulted in decreased kidney retention, improved signal-to-noise ratio, and enhanced PET image quality (FIG. 9B-FIG. 9C). This outcome aligned with the previous finding that PEGylation reduces kidney retention of PET tracers while enhancing specific uptake and circulation half-life (Rashidian et al., J. Exp. Med. 214(8):2243-2255 (2017), Rashidian et al., Proc. Natl. Acad. Sci. U.S.A 116(34):16971-16980 (2019)). PEGylation with a 20 kDa PEG molecules brings the tracer's total size to around 52 kDa, which is below the glomerular filtration size of −60 kDa. The −52 kDa PEGylated CD19 tracer demonstrated efficient clearance from the circulation, enabling next-day PET imaging (FIG. 9D). Consequently, subsequent experiments were conducted using the CD19 tracer with the site-specific installation of a 20 kDa PEG (PEG20). Remarkably, the results demonstrated that mice treated with CD19 CAR T cells exhibited a specific PET signal in the spleen and bone marrow visualized on in vivo images and was confirmed in ex vivo images (FIG. 9B-FIG. 9C).

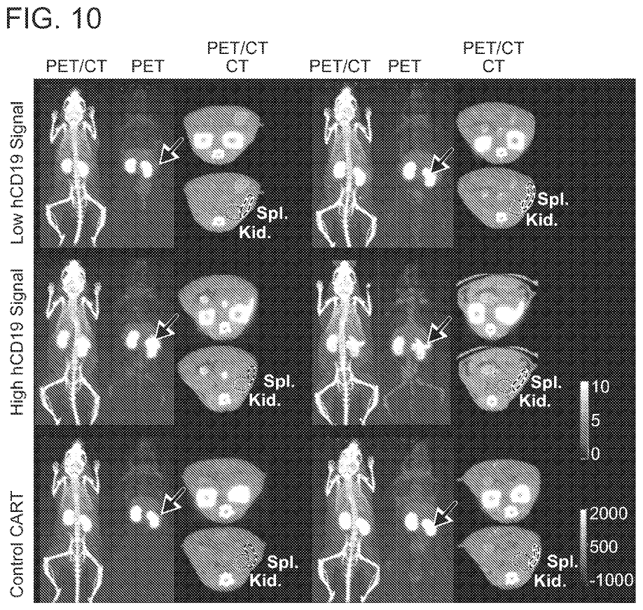

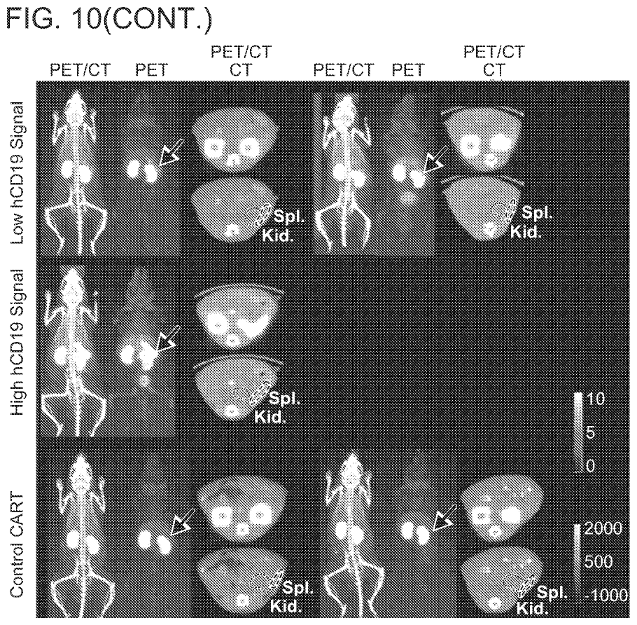

Following optimization of imaging quality, imaging on a larger group of mice was conducted using the ⁸⁹Zr-DFO-PEG20-labeled CD19 tracer. Mice were intravenously injected with 3×10⁶ hCD19⁺ B-ALL cells, followed by the administration of 10×10⁶ CD19 or control (EGFRvIII) CAR T cells three days later (FIG. 4A). Two days post-CAR T cell injection, the ⁸⁹Zr-DFO-PEG20 labeled CD19 tracer was intravenously administered (~50 μCi of activity, ~5 μg of the tracer, per mouse). The tracer's clearance from the blood was monitored within the first 48 hours following injection (FIG. 9D). During this evaluation, all mice displayed rapid tracer clearance from the bloodstream, with approximately half of the tracer being eliminated within the initial ~1-2 hours post-injection. Twenty-four hours post-injection of the ⁸⁹Zr-DFO-PEG20-labeled CD19 tracer, the animals were subjected to PET imaging with concurrent computed tomography (CT) imaging to localize PET signal to anatomic structures. Distinct PET signals were observed in the spleen and bone marrow of mice injected with CD19 CAR T cells, which are the primary sites of leukemic burden. In contrast, these signals were absent in mice administered with control CAR T cells (FIG. 4B and FIG. 10).

Quantitative analysis of the PET images unveiled variations in PET signals within the spleens of CD19 CAR T cell-treated mice (FIG. 4C). While certain mice displayed standardized uptake values (SUV) below 10, approximately half exhibited SUVs exceeding 10. In contrast, the mean SUV value for mice injected with control CAR T cells in their spleen was approximately 1.88. Consequently, the CD19 CAR T cell-treated mice were categorized into two groups: those with “high CD19⁻ PET signal” (SUV>10) and those with “low CD19⁻ PET signal” (SUV<10). The survival of these mice were monitored during the PET imaging period. Notably, an extension in survival was observed among the mice belonging to the “high CD19⁻ PET signal” group compared to those with “low CD19⁻ PET signal” (FIG. 4D). In fact, the CD19 CAR T cell-treated mice in the “low CD19⁻ PET signal” group survived to a similar extent as control CAR T cell-treated mice.

When mice became moribund, the biodistribution of the Zr⁸⁹-CD19 tracer was analyzed by measuring the amount of radioactivity left in each tissue (FIG. 9E). At the time of death, the ⁸⁹Zr-CD19 tracer signal in the spleen of mice injected with CD19 CAR T cells was still significantly (p=0.0234) higher than in mice injected with control CAR T cells (FIG. 9E).

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present disclosure as defined by the appended claims.

1. A radiolabeled tracer, comprising:
an ectodomain of an antigen present on a cancer cell; and
a radioactive isotope detectable via positron emission tomography (PET) or single-photon emission computerized tomography (SPECT) connected to the ectodomain.

2. The radiolabeled tracer of claim 1, wherein the ectodomain is obtainable from AFP, AXL, B4GALNT1, B-cell maturation antigen (BCMA), CA9, CD5, CD7, CD19, CD22, CD23, CD33, CD34, CD38, CD44, CD52, CD70, CD80, CD86, CD123, CD133, CD174, CD274, CD276, CDS, Cancer/Testis Antigen 1B (CTAG1B), CEACAM5, CLEC12A, claudin 18.2 (CLDN 18.2), CSPG4, DLL3, EGFR, EPCAM, EPHA2, ERBB2, FAP, FOLH1, FOLR1, GD2, GPC3, GPRC5D, GPNMB, HER2, HPV E7, IL1RAP, IL3RA, IL13Ru2, KDR, KIT, KLRK1, L1CAM, MAGEA1, MAGEA4, MET, MME, MSLN, MUC1, MUC16, MS4A1, NCAM1, PDCD1, PMEL, PROM1, PSCA, ROR1, ROR2, SDC1, SLAM7, TEM1, TNF Receptor Superfamily Member (TNFRSF) 8, TNFRSF10B, TNFRSF13C, TNFRSF17, ULBP1, or ULBP2.

3. The radiolabeled tracer of claim 2, wherein the ectodomain is a BCMA ectodomain.

4. The radiolabeled tracer of claim 3, wherein the ectodomain comprises the amino acid sequence SEQ ID NO: 1.

5. The radiolabeled tracer of claim 2, wherein the ectodomain is a CD19 ectodomain; and
wherein the ectodomain comprises an amino acid sequence having at least about 85% sequence identity with SEQ ID NO: 2; or
wherein the ectodomain comprises the amino acid sequence of SEQ ID NO: 2; or
wherein the ectodomain comprises the amino acid sequence of SEQ ID NO: 3 or SEQ ID NO: 4.

6.-8. (canceled)

9. The radiolabeled tracer of claim 2, wherein the ectodomain is a CD20 ectodomain, a SLAMF7 ectodomain, a PD-1 ectodomain, a KIT ectodomain, or a CD38 ectodomain.

10. The radiolabeled tracer of claim 9, wherein the ectodomain is a CD20 ectodomain and comprises the amino acid sequence SEQ ID NO: 5; or
wherein the ectodomain is a SLAMF7 ectodomain and comprises the amino acid sequence SEQ ID NO: 6, or
wherein the ectodomain is a PD-1 ectodomain and comprises the amino acid sequence SEQ ID NO: 7; or
wherein the ectodomain is a KIT ectodomain and comprises the amino acid sequence SEQ ID NO: 8; or
wherein the ectodomain is a CD38 ectodomain and comprises the amino acid sequence SEQ ID NO: 9.

11.-18. (canceled)

19. The radiolabel tracer of claim 1, further comprising a linker disposed between the radioactive isotope and the ectodomain.

20. The radiolabeled tracer of claim 19, further comprising a constant heavy (CH) 3 domain disposed between the ectodomain and the linker.

21. The radiolabeled tracer of claim 20, wherein the CH3 domain comprises the amino acid sequence SEQ ID NO: 10: or
wherein the radioactive isotope is directly connected to an amino acid residue of the ectodomain or the CH3 domain; or
wherein the radioactive isotope is covalently bonded to a tyrosine sidechain of the ectodomain.

22.-23. (canceled)

24. The radiolabeled tracer of claim 21, wherein the linker comprises LPXTGGG-R1 (SEQ ID NO: 11), wherein X is any amino acid and R1 is any biomolecule; or
wherein the linker or R1 comprises desferrioxamine (DFO), dodecane tetraacetic acid (DOTA), 3,4,3-(LI-1,2-HOPO), 1,4,7-Triazacyclononane-1,4,7-triacetic acid (NOTA), 1,4,7-triazacyclononane,1-glutaric acid-4,7-acetic acid (NODAGA), or 3,6,9,15-Tetraazabicyclo[9.3.1]pentadeca-1(15),11,13-triene-3,6,9-triacetic acid (PCTA); or
wherein R1 is transcyclooctene, azide, alkyne, or tetrazine.

25.-26. (canceled)

27. The radiolabeled tracer of claim 1, wherein the radioactive isotope is zirconium-89 (⁸⁹Zr), fluorine-18 (¹⁸F), iodine-124 (¹²⁴I), copper-64 (⁶⁴Cu), gallium-68 (⁶⁸Ga), yttrium-90 (⁹⁰Y), indium-111 (¹¹¹In), or technetium-99 m (^{99m}Tc), iodine-123 (¹²³I), or iodine-131 (¹³¹I).

28. The radiolabel tracer of claim 1, wherein the radioactive isotope is detectable via PET.

29. The radiolabeled tracer of claim 28, wherein the radioactive isotope is ⁸⁹Zr or ⁶⁴Cu.

30. (canceled)

31. The radiolabel tracer of claim 1, wherein the radioactive isotope is detectable via SPECT.

32. The radiolabeled tracer of claim 31, wherein the radioactive isotope is ¹¹¹In or ^{99m}Tc.

33. (canceled)

34. The radiolabeled tracer of claim 1, which is conjugated to a polyethylene glycol (PEG) molecule.

35. The radiolabeled tracer of claim 34, wherein the PEG molecule has a molecular weight of about 10 kilodaltons (kDa) or about 20 kDa and is conjugated to at least one of the ectodomain, linker, and dimerization domain.

36. (canceled)

37. The radiolabeled tracer of claim 19, which is in the form of a dimer.

38. A diagnostic composition comprising an effective amount of the radiolabeled tracer of claim 1, and a pharmaceutically acceptable carrier.

39. A method of detecting chimeric antigen receptor (CAR) immune cells in a subject, comprising:
administering to the subject the diagnostic composition of claim 38;
wherein prior to the administering of the diagnostic composition, the subject had been administered an effective number of immune cells comprising a CAR that comprises an extracellular domain that binds the ectodomain of the radiolabeled tracer, a transmembrane domain, and an intracellular domain comprising a stimulatory domain (CAR immune cells); and
producing a first image of a body area of the subject by PET or SPECT imaging; wherein
detecting the radioactive isotope in the body area is an indication of the CAR immune cells in the body area.

40. The method of claim 39, further comprising:
administering to the subject, an effective amount of the diagnostic composition after the first image is produced; and
producing a second image of the body area of the subject;

41. The method of claim 40, comprising producing the second image at least one month or at least six months after producing the first image or producing the first and the second images by PET imaging.

42.-43. (canceled)

44. The method of claim 40, wherein the first and the second images are performed by PET-computed tomography (PET-CT), PET-magnetic resonance imaging (PET-MRI), or SPECT imaging.

45. (canceled)

46. The method of claim 38, wherein the body area includes bone marrow, lymph node tissue, spleen tissue, lung tissue, liver tissue, peritoneum, tumor tissue, or a combination thereof: or
wherein the body area includes the entire body.

47. The method of claim 46, wherein the tissue is tumor tissue.

48. (canceled)

49. A kit, comprising:
a) an effective amount of the radiolabeled tracer of claim 1; and
b) printed instructions for use in administering to a subject having received or receiving an administration of an effective number of CAR immune cells.

50. The kit of claim 49, wherein the effective amount of the radiolabeled tracer is formulated in a pharmaceutically acceptable carrier.

51. A method of treating cancer in a subject comprising:
determining the presence of CAR immune cells in a body area of the subject by the method of claim 39; and
administering an appropriate therapy to the subject following a determination that the presence or number of CAR immune cells is suboptimal.

52. The method of claim 51, wherein the determination is assessed on the basis of CAR immune cells detected in spleen, lymph nodes, bone marrow or tumor tissue; or
wherein the lack of substantive radioactive isotope detection in the body area indicates that the presence or number of CAR immune cells is suboptimal; or
wherein the appropriate therapy comprises immunotherapy, immune cell regulatory protein inhibition, TGF-b inhibition, or a combination thereof: or
wherein the cancer is leukemia or pancreatic cancer.

53-55. (canceled)
